SEQUENCE LISTING (6 sequences)

SEQ ID NO: 1 - protein, 70 aa, Pyrococcus horikoshii
MWMMGELPIAPVDRLIRKAGAERVSEQAAKVLAEYLEEYAIEIAKKAVEFARHAGRKTVKVEDIKLAIKS

SEQ ID NO: 2 - protein, 67 aa, synthetic construct
  REGION 1..67: note = Synthetic
MAELPIAPIDRLIRKAGAERVSEEAAKVLAEYLEEYAIELSKKAVEFARHAGRKTVKAEDIKLAIKA

SEQ ID NO: 3 - DNA, 213 bp, Pyrococcus horikoshii
  source 1..213: mol_type = unassigned DNA
atgtggatgatgggagaattaccaattgccccagttgacagacttattagaaaggcaggtgccgagagggtcagcgagcaagctgcaaaggttctcgccgagtaccttgaagagtacgcaatcgagattgcaaagaaggccgttgagtttgcaaggcacgctggaagaaagaccgtcaaggttgaggacattaagctcgctattaagagctga

SEQ ID NO: 4 - DNA, 257 bp, Pyrococcus horikoshii
  source 1..257: mol_type = unassigned DNA
ccatggatcaccatcatcatcaccatcatgatatccatatgtggatgatgggagaattaccaattgccccagttgacagacttattagaaaggcaggtgccgagagggtcagcgagcaagctgcaaaggttctcgccgagtaccttgaagagtacgcaatcgagattgcaaagaaggccgttgagtttgcaaggcacgctggaagaaagaccgtcaaggttgaggacattaagctcgctattaagagctgactcgag

SEQ ID NO: 5 - DNA, 257 bp, synthetic construct
  misc_feature 1..257: note = Synthetic
  source 1..257: mol_type = other DNA
ccatggatcaccatcatcatcaccatcatgatatccatatgtggatgatgggtgaactgccaatcgcgccggtcgaccgtctgatccgtaaagcgggtgctgaacgcgtcagcgagcaggcagccaaagtgcttgcagaatacctggaagagtatgctatcgagatcgcaaagaaagctgttgagtttgcccgacatgcgggtcgtaagaccgtgaaagttgaggatatcaagctggcaattaaaagttgactcgag

SEQ ID NO: 6 - protein, 464 aa, synthetic construct
  REGION 1..464: note = Synthetic
HHHHHHHMNNAQWAKSTFGQADLGDPRRTTRLVKLAETLANDPGKPFVSITQSKADAEGAYRFIRNEHVNADAIAKAGYLVTAAQAAKHNLLLALEDTTAITYSHRSVRDELGHVNQGNNYRGILAHSVLLFAPEQQELVGLIEQSRWTRDISTRGKKHVRTQTPYEEKESFKWQSASVNLSARLGTKMADVISVCDREADIYEYLQYKLSKQHRFVVRSMQSRHIEQSEQKLYDYAAGLESAGQKQIHIAQKGGRKARTATVDIVFAPVTLQVPANKRGESLSLYYVGCEERADDKNALNWHLLTTEPVQSKADALNIIRYYEHRWLVEEYHKAWKTDGTDIENARLQSKDNIERLVTISAFIAVRIVQLKFAREQPDEISCEQVLSPKAWKLLWIKRVSRTLPDTVPSMKWAYTELAKLGGWKDTKQTGKASVKVLWQGWFKLQTILEGYDLAKSLEADLLE

(19) United States
(12) Patent Application Publication
(10) Pub. No.: US 2025/0368983 A1
(43) Pub. Date: Dec. 4, 2025

(54) CONTROLLING FOR TAGMENTATION SEQUENCING LIBRARY INSERT SIZE USING ARCHAEAL HISTONE-LIKE PROTEINS

(71) Applicant: Kapa Biosystems, Inc., Wilmington, MA (US)

(72) Inventors: Taariq Firfirey, Cape Town (ZA); Etienne Slabbert, Western Cape (ZA)

(21) Appl. No.: 18/876,416

(22) PCT Filed: Jun. 30, 2023
(86) PCT No.: PCT/EP2023/067959

§ 371 Date: Dec. 18, 2024

Related U.S. Application Data
(60) Provisional application No. 63/367,371, filed on Jun. 30, 2022.

(51) Int. Cl. C12N 15/10; C07K 14/195; C12Q 1/48; C12Q 1/6806

(52) U.S. Cl. CPC C12N 15/1065; C07K 14/195; C12Q 1/485; C12Q 1/6806; G01N 2333/91245

(57) ABSTRACT

The present disclosure provides compositions and kits for the tagmentation of double stranded DNA. In some embodiments, the compositions and kits for the tagmentation of double stranded DNA include one or more histone-like proteins and/or one or more transposition systems. The present disclosure also provides methods for the tagmentation of double stranded DNA in the presence of one or more histone-like proteins.

### BACKGROUND OF THE DISCLOSURE

The advent of single cell genome amplification techniques and next generation sequencing methods have led to breakthroughs in the ability to sequence the genome and transcriptome of individual biological cells. Massively parallel DNA sequencing of thousands of samples in a single instrument-run is now possible, but the preparation of the individual sequencing libraries is expensive and time-consuming. Library preparation is an essential process preceding sequencing itself, and comprises several aspects that affect the efficiency of sequencing. Library preparation “typically involves the following main steps: fragmentation of the input DNA, end-repair and A-tailing of the DNA fragments, ligation of indexed sequencing adapters and optional amplification of the ligated products.” (Ribarska et. al., ‘Optimization of enzymatic fragmentation is crucial to maximize genome coverage: a comparison of library preparation methods for Illumina sequencing,’ BMC Genomics. 2022; 23:92).

One of the major bottlenecks to sample preparation is DNA fragmentation. The size of the DNA fragments generated depend on the sequencing platform being used, and can range from several hundred base pairs for short read sequencing technologies (e.g., Illumina®, Ion Torrent™) to >10 kb pieces for long read sequencing technologies (e.g., Pacific Biosciences® and Oxford Nanopore Technologies®). Methods for fragmenting DNA are broadly split into two basic categories: mechanical and enzyme-based. Mechanical shearing methods include acoustic shearing, hydrodynamic shearing and nebulization, while enzyme-based methods include transposons, restriction enzymes and nicking enzymes. Although many different options exist to fragment DNA, final fragment size, amount of starting material, upfront capital investment, and scalability must be considered when choosing a fragmentation method. Critically, in order to be useful for next generation sequencing, the method used must shear the DNA sufficiently randomly, so that the library being sequenced is fully representative of the original sample.

Tagmentation is a process that combines fragmentation and an adapter incorporation step. The term “tagmenting” as used herein refers to the transposase-catalyzed combined fragmentation of a double-stranded DNA sample and tagging of the fragments with sequences that are adjacent to a transposon end sequence. A hyperactive variant of the bacterial Tn5 transposase that mediates the fragmentation of double-stranded DNA and ligates synthetic oligonucleotides has been widely employed in next-generation sequencing (NGS). Its utility in generating libraries for NGS systems was first described in a paper by Andrew Adey et al. in 2010 (Adey et al., “Rapid, low-input, low-bias construction of shotgun fragment libraries by high-density in vitro transposition,” Genome Biol 11: R119, 2010, the disclosure of which is hereby incorporated by reference herein in its entirety). In commercially available products such as Nextera from Illumina, and MuSeek from Thermo Scientific, the transposase inserts NGS system-specific adaptor oligonucleotides in the double stranded DNA sample. Such a simple one-step tagmentation reaction has greatly simplified the process of preparing libraries for sequencing, shortening the workflow time and lowering costs.

### BRIEF SUMMARY OF THE DISCLOSURE

The approach of sequencing using tagmentation involves the fragmentation of double-stranded DNA while adding universal overhangs. As noted above, the workflow allows for the quick generation of sequencing libraries because of this combined fragmentation and tagging step. The sequencing library preparation process is, however, highly sensitive to the input DNA concentration and/or the input transposition system concentration. As such, precise quantification of the input DNA concentration and/or the transposition system concentration is necessary to control fragment size. Applicant has unexpectedly discovered that tagmentation in the presence of one or more histone-like proteins mitigates the need for precise quantification of the input DNA concentration and/or the transposition system concentration. In addition, Applicant has discovered that tagmentation in the presence of one or more histone-like proteins allows for precise control over the DNA fragment size distribution. Moreover, Applicant has discovered that tagmentation in the presence of one or more histone-like proteins allows for better control over the tagmentation fragment size, facilitating greater utility of tagmentation in applications requiring longer fragment inserts.

A first aspect of the present disclosure is a composition comprising a histone-like protein and a transposition system. In some embodiments, the histone-like protein is derived from a thermophilic or hyperthermopohilic Archaea. In some embodiments, the histone-like protein is an archaeal histone-like protein derived from one of a Thermococcus or a Pyrococcus. In some embodiments, histone-like protein comprises an amino acid sequence having 85% identity to any one of SEQ ID NOS: 1 to 2. In some embodiments, the histone-like protein comprises an amino acid sequence having 90% identity to any one of SEQ ID NOS: 1 to 2. In some embodiments, the histone-like protein comprises an amino acid sequence having 95% identity to any one of SEQ ID NOS: 1 to 2. In some embodiments, the histone-like protein comprises any one of SEQ ID NOS: 1 to 2. In some embodiments, a concentration of the histone-like protein in the composition ranges from between about 2.5 ng/μL to about 25 ng/μL. In some embodiments, a concentration of the histone-like protein in the composition ranges from between about 5 ng/μL to about 20 ng/μL. In some embodiments, a concentration of the histone-like protein in the composition ranges from between about 7.5 ng/μL to about 20 ng/μL.

In some embodiments, the transposition system includes a transposase, and adapters. In some embodiments, the transposition system comprises a hyperactive Tn5 transposase and a Tn5-type transposase recognition site. In some embodiments, the transposition system comprises a hyperactive Tn5 transposase, a Tn5-type transposase recognition site, and one or more oligonucleotides. In some embodiments, a concentration of the transposition system in the composition ranges from between about 150 ng/μL to about 200 ng/μL. In some embodiments, a concentration of the transposition system ranges in the composition is about 180 ng/μL.

In some embodiments, the composition further comprises a nucleic acid sequence, such as DNA, ctDNA, double-stranded DNA, etc. In some embodiments, the double-stranded DNA is derived from a human subject. In some embodiments, the double-stranded DNA is derived from a tumor sample. In some embodiments, concentration of the double-stranded DNA in the composition ranges from between about 2 ng/μL to about 8 ng/μL. In some embodiments, a concentration of the double-stranded DNA in the composition ranges from between about 3 ng/μL to about 7 ng/μL. In some embodiments, a concentration of the double-stranded DNA in the composition is about 5 ng/μL. In some embodiments, a ratio of a concentration of the histone-like protein to a concentration of DNA in the composition ranges from between about 0.5:1 to about 5:1. In some embodiments, a ratio of a concentration of the histone-like protein to a concentration of DNA in the composition ranges from between about 1:1 to about 4:1. In some embodiments, a ratio of a concentration of the histone-like protein to a concentration of DNA in the composition ranges from between about 1.5:1 to about 3:1.

In some embodiments, the composition further comprises a divalent cation. In some embodiments, the divalent cation is selected from the group consisting of Co2+, Mn2+, Mg2+, Cd2+, and Ca2+. In some embodiments, the divalent cation is Mn2+. In some embodiments, the composition further a LMW buffer. In some embodiments, the LMW buffer comprises tris-acetate, glycerol, and DMSO.

A second aspect of the present disclosure is a composition comprising a histone-like protein, a transposition system, and double stranded DNA. In some embodiments, the histone-like protein is derived from a thermophilic or hyperthermopohilic Archaea. In some embodiments, the histone-like protein is an archaeal histone-like protein derived from one of a Thermococcus or a Pyrococcus. In some embodiments, histone-like protein comprises an amino acid sequence having 85% identity to any one of SEQ ID NOS: 1 to 2. In some embodiments, the histone-like protein comprises an amino acid sequence having 90% identity to any one of SEQ ID NOS: 1 to 2. In some embodiments, the histone-like protein comprises an amino acid sequence having 95% identity to any one of SEQ ID NOS: 1 to 2. In some embodiments, the histone-like protein comprises any one of SEQ ID NOS: 1 to 2. In some embodiments, a concentration of the histone-like protein in the composition ranges from between about 2.5 ng/μL to about 25 ngμL. In some embodiments, a concentration of the histone-like protein in the composition ranges from between about 5 ng/μL to about 20 ng/μL. In some embodiments, a concentration of the histone-like protein in the composition ranges from between about 7.5 ng/μL to about 20 ng/μL.

In some embodiments, the transposition system comprises a transposase, a transposon, and adapters. In some embodiments, the transposition system comprises TnAa. In some embodiments, the transposition system comprises a hyperactive Tn5 transposase and a Tn5-type transposase recognition site. In some embodiments, the transposition system comprises a hyperactive Tn5 transposase, a Tn5-type transposase recognition site, and one or more oligonucleotides. In some embodiments, a concentration of the transposition system in the composition ranges from between about 150 ng/μL to about 200 ng/μL. In some embodiments, a concentration of the transposition system ranges in the composition is about 180 ng/μL.

In some embodiments, concentration of the double-stranded DNA in the composition ranges from between about 2 ng/μL to about 8 ng/μL. In some embodiments, a concentration of the double-stranded DNA in the composition ranges from between about 3 ng/μL to about 7 ng/μL. In some embodiments, a concentration of the double-stranded DNA in the composition is about 5 ng/μL. In some embodiments, a ratio of a concentration of the histone-like protein to a concentration of DNA in the composition ranges from between about 0.5:1 to about 5:1. In some embodiments, a ratio of a concentration of the histone-like protein to a concentration of DNA in the composition ranges from between about 1:1 to about 4:1. In some embodiments, a ratio of a concentration of the histone-like protein to a concentration of DNA in the composition ranges from between about 1.5:1 to about 3:1

In some embodiments, the composition further comprises a divalent cation. In some embodiments, the divalent cation is selected from the group consisting of Co2+, Mn2+, Mg2+, Cd2+, and Ca2+. In some embodiments, the divalent cation is Mn2+. In some embodiments, the composition further comprises a LMW buffer. In some embodiments, the LMW buffer comprises tris-acetate, glycerol, and DMSO.

A third aspect of the present disclosure is a composition comprising a histone-like protein, a transposition system, double stranded DNA, and a divalent cation (e.g., Co2+, Mn2+, Mg2+, Cd2+, and Ca2+). In some embodiments, the histone-like protein is derived from a thermophilic or hyperthermopohilic Archaea. In some embodiments, the histone-like protein is an archaeal histone-like protein derived from one of a Thermococcus or a Pyrococcus. In some embodiments, histone-like protein comprises an amino acid sequence having 85% identity to any one of SEQ ID NOS: 1 to 2. In some embodiments, the histone-like protein comprises an amino acid sequence having 90% identity to any one of SEQ ID NOS: 1 to 2. In some embodiments, the histone-like protein comprises an amino acid sequence having 95% identity to any one of SEQ ID NOS: 1 to 2. In some embodiments, the histone-like protein comprises any one of SEQ ID NOS: 1 to 2. In some embodiments, a concentration of the histone-like protein in the composition ranges from between about 2.5 ng/μL to about 25 ngμL. In some embodiments, a concentration of the histone-like protein in the composition ranges from between about 5 ng/μL to about 20 ng/μL. In some embodiments, a concentration of the histone-like protein in the composition ranges from between about 7.5 ng/μL to about 20 ng/μL.

In some embodiments, the transposition system comprises a transposase, a transposon, and adapters. In some embodiments, the transposition system comprises TnAa. In some embodiments, the transposition system comprises a hyperactive Tn5 transposase and a Tn5-type transposase recognition site. In some embodiments, the transposition system comprises a hyperactive Tn5 transposase, a Tn5-type transposase recognition site, and one or more oligonucleotides. In some embodiments, a concentration of the transposition system in the composition ranges from between about 150 ng/μL to about 200 ng/μL. In some embodiments, a concentration of the transposition system ranges in the composition is about 180 ng/μL.

In some embodiments, concentration of the double-stranded DNA in the composition ranges from between about 2 ng/μL to about 8 ng/μL. In some embodiments, a concentration of the double-stranded DNA in the composition ranges from between about 3 ng/μL to about 7 ng/μL. In some embodiments, a concentration of the double-stranded DNA in the composition is about 5 ng/μL. In some embodiments, a ratio of a concentration of the histone-like protein to a concentration of DNA in the composition ranges from between about 0.5:1 to about 5:1. In some embodiments, a ratio of a concentration of the histone-like protein to a concentration of DNA in the composition ranges from between about 1:1 to about 4:1. In some embodiments, a ratio of a concentration of the histone-like protein to a concentration of DNA in the composition ranges from between about 1.5:1 to about 3:1.

A fourth aspect of the present disclosure is a kit comprising a first container comprising a histone-like protein; and a second container comprising a transposition system. In some embodiments, the histone-like protein is derived from a thermophilic or hyperthermopohilic Archaea. In some embodiments, the histone-like protein is an archaeal histone-like protein derived from one of a Thermococcus or a Pyrococcus. In some embodiments, the histone-like protein comprises an amino acid sequence having 85% identity to any one of SEQ ID NOS: 1 to 2. In some embodiments, the histone-like protein comprises an amino acid sequence having 90% identity to any one of SEQ ID NOS: 1 to 2. In some embodiments, the histone-like protein comprises an amino acid sequence having 95% identity to any one of SEQ ID NOS: 1 to 2. In some embodiments, the histone-like protein comprises any one of SEQ ID NOS: 1 to 2. In some embodiments, a concentration of the histone-like protein in the composition ranges from between about 2.5 ng/μL to about 25 ngμL. In some embodiments, a concentration of the histone-like protein in the first container ranges from between about 5 ng/μL to about 20 ng/μL. In some embodiments, a concentration of the histone-like protein in the first container ranges from between about 7.5 ng/μL to about 20 ng/μL.

In some embodiments, the transposition system includes a transposase, and adapters. In some embodiments, the transposition system comprises a hyperactive Tn5 transposase and a Tn5-type transposase recognition site. In some embodiments, the transposition system comprises a hyperactive Tn5 transposase, a Tn5-type transposase recognition site, and one or more oligonucleotides. In some embodiments, a concentration of the transposition system in the second container ranges from between about 150 ng/μL to about 200 ng/μL. In some embodiments, a concentration of the transposition system ranges in the second container is about 180 ng/μL. In some embodiments, the kit further comprises a third container comprising a divalent cation and/or one or more PCR reagents (e.g., a polymerase).

A fifth aspect of the present disclosure is a method of tagmenting double-strand DNA comprising: (i) obtaining a sample comprising double-stranded DNA; (ii) introducing a fragmentation composition comprising a histone-like protein and a transposition system to the obtained sample to provide a fragmentation reaction mixture; (iii) heating the fragmentation reaction mixture for a predetermined amount of time at a predetermined temperature; and (iv) isolating the tagmented DNA from the fragmentation reaction mixture. In some embodiments, the histone-like protein is derived from a thermophilic or hyperthermopohilic Archaea. In some embodiments, the histone-like protein comprises an amino acid sequence having 85% identity to any one of SEQ ID NOS: 1 to 2. In some embodiments, the histone-like protein comprises an amino acid sequence having 90% identity to any one of SEQ ID NOS: 1 to 2. In some embodiments, the histone-like protein comprises an amino acid sequence having 95% identity to any one of SEQ ID NOS: 1 to 2. In some embodiments, the histone-like protein comprises any one of SEQ ID NOS: 1 to 2. In some embodiments, a concentration of the histone-like protein in the fragmentation reaction mixture ranges from between about 2.5 ng/μL to about 25 ngμL. In some embodiments, a concentration of the histone-like protein in the fragmentation reaction mixture ranges from between about 5 ng/μL to about 20 ng/μL. In some embodiments, a concentration of the histone-like protein in the fragmentation reaction mixture ranges from between about 7.5 ng/μL to about 20 ng/μL.

In some embodiments, the transposition system comprises a transposase, a transposon, and adapters. In some embodiments, the transposition system comprises a hyperactive Tn5 transposase and a Tn5-type transposase recognition site. In some embodiments, the transposition system comprises a hyperactive Tn5 transposase, a Tn5-type transposase recognition site, and one or more oligonucleotides. In some embodiments, a concentration of the transposase in the fragmentation reaction mixture ranges from between about 150 ng/μL to about 200 ng/μL. In some embodiments, a concentration of the transposase ranges in the fragmentation reaction mixture is about 180 ng/μL.

In some embodiments, concentration of the double-stranded DNA in the composition ranges from between about 2 ng/μL to about 8 ng/μL. In some embodiments, a concentration of the double-stranded DNA in the composition ranges from between about 3 ng/μL to about 7 ng/μL. In some embodiments, a concentration of the double-stranded DNA in the composition is about 5 ng/μL. In some embodiments, a ratio of a concentration of the histone-like protein to a concentration of the double stranded DNA in the fragmentation reaction mixture ranges from between about 0.5:1 to about 5:1. In some embodiments, a ratio of a concentration of the histone-like protein to a concentration of the double stranded DNA in the fragmentation reaction mixture ranges from between about 1:1 to about 4:1. In some embodiments, a ratio of a concentration of the histone-like protein to a concentration of the double stranded DNA in the fragmentation reaction mixture ranges from between about 1.5:1 to about 3:1.

In some embodiments, the predetermined time ranges from between about 2 minutes to about 10 minutes. In some embodiments, the predetermined time ranges from between about 3 minutes to about 7 minutes. In some embodiments, the predetermined time is about 5 minutes. In some embodiments, the predetermined temperature is about 40° C. to about 60° C. In some embodiments, the predetermined temperature is about 45° C. to about 55° C. In some embodiments, the predetermined temperature is about 50° C. to about 55° C.

In some embodiments, the method further comprises introducing a stop solution to the reaction mixture. In some embodiments, the stop solution comprises SDS.

In some embodiments, the isolation of the tagmented DNA comprises: (a) capturing generated DNA fragments onto beads; (b) flushing impurities from the reaction mixture; and (c) eluting the captured DNA fragments from the beads. In some embodiments, the isolated tagmented DNA has a size distribution ranging from between about 250 bp to about 300 bp. In some embodiments, the method further comprises amplifying the isolated double-stranded DNA fragments to provide a plurality of amplicons. In some embodiments, the method further comprises sequencing the plurality of amplicons.

A sixth aspect of the present disclosure is a method for processing a sample including genomic material comprising: (i) obtaining a tagmentation reaction mixture including a transposition system and optionally a buffer; (ii) introducing to the tagmentation reaction mixture a solution comprising one or more nucleosome-like structures to provide a fragmentation reaction mixture, wherein the one or more nucleosome-like structures include double-stranded DNA wound or wrapped around one or more histone-like proteins; and (iii) heating the fragmentation reaction mixture to a predetermined temperature for a predetermined amount of time. In some embodiments, the double-stranded DNA and the histone-like protein are first mixed together to form a DNA-histone-like protein solution, and then the DNA-histone-like protein solution is added to the tagmentation reaction mixture to provide the fragmentation reaction mixture. In some embodiments, the histone-like protein is derived from a thermophilic or hyperthermopohilic Archaea. In some embodiments, the histone-like protein is an archaeal histone-like protein derived from one of a Thermococcus or a Pyrococcus. In some embodiments, the histone-like protein comprises an amino acid sequence having 85% identity to any one of SEQ ID NOS: 1 to 2. In some embodiments, the histone-like protein comprises an amino acid sequence having 90% identity to any one of SEQ ID NOS: 1 to 2. In some embodiments, the histone-like protein comprises an amino acid sequence having 95% identity to any one of SEQ ID NOS: 1 to 2. In some embodiments, the histone-like protein comprises any one of SEQ ID NOS: 1 to 2.

In some embodiments, concentration of the double-stranded DNA in the composition ranges from between about 2 ng/μL to about 8 ng/μL. In some embodiments, a concentration of the double-stranded DNA in the composition ranges from between about 3 ng/μL to about 7 ng/μL. In some embodiments, a concentration of the double-stranded DNA in the composition is about 5 ng/μL. In some embodiments, a ratio of a concentration of the histone-like protein in the tagmentation reaction mixture to a concentration of the double-stranded DNA in the fragmentation reaction mixture ranges from between about 1:1 to about 4:1. In some embodiments, the ratio ranges from between about 1.5:1 to about 3:1.

A seventh aspect of the present disclosure is a method for processing a sample including genomic material, comprising: (i) obtaining a sample in a reaction vessel, the sample including double stranded DNA material; (ii) introducing a histone-like protein to the reaction vessel to provide a DNA-histone-like protein solution; (iii) introducing a transposition system to the DNA-histone-like protein solution to provide a fragmentation reaction mixture; and (iv) heating the sample the fragmentation reaction mixture to a predetermined temperature for a predetermined amount of time. In some embodiments, the transposition system comprises TnAa or a hyperactive mutant of Tn5 transposase and oligonucleotide material. In some embodiments, the oligonucleotide material includes synthetic oligonucleotides. In some embodiments, the histone-like protein is derived from a thermophilic or hyperthermopohilic Archaea. In some embodiments, the histone-like protein comprises an amino acid sequence having 85% identity to any one of SEQ ID NOS: 1 to 2. In some embodiments, the histone-like protein comprises an amino acid sequence having 90% identity to any one of SEQ ID NOS: 1 to 2. In some embodiments, the histone-like protein comprises an amino acid sequence having 95% identity to any one of SEQ ID NOS: 1 to 2. In some embodiments, the histone-like protein comprises any one of SEQ ID NOS: 1 to 2.

In some embodiments, concentration of the double-stranded DNA in the composition ranges from between about 2 ng/μL to about 8 ng/μL. In some embodiments, a concentration of the double-stranded DNA in the composition ranges from between about 3 ng/μL to about 7 ng/μL. In some embodiments, a concentration of the double-stranded DNA in the composition is about 5 ng/μL.

In some embodiments, a concentration of the histone-like protein in the fragmentation reaction mixture ranges from between about 2.5 ng/μL to about 25 ngμL. In some embodiments, a concentration of the histone-like protein in the fragmentation reaction mixture ranges from between about 5 ng/μL to about 20 ng/μL. In some embodiments, a concentration of the histone-like protein in the fragmentation reaction mixture ranges from between about 7.5 ng/μL to about 20 ng/μL. In some embodiments, a concentration of the histone-like protein in the fragmentation reaction mixture ranges from between about 10 ng/μL to about 20 ng/μL. In some embodiments, a concentration of the histone-like protein in the reaction fragmentation reaction mixture ranges from between about 10 ng/μL to about 15 ng/μL. In some embodiments, a concentration of the histone-like protein in the fragmentation reaction mixture ranges from between about 15 ng/μL to about 20 ng/μL.

In some embodiments, wherein the predetermined time ranges from between about 2 minutes to about 10 minutes. In some embodiments, the predetermined time ranges from between about 3 minutes to about 7 minutes. In some embodiments, the predetermined time is about 5 minutes. In some embodiments, the predetermined temperature is about 40° C. to about 60° C. In some embodiments, the predetermined temperature is about 45° C. to about 55° C. In some embodiments, the predetermined temperature is about 50° C. to about 55° C.

In some embodiments, the method further comprises isolating tagmented DNA from the fragmentation reaction mixture. In some embodiments, the isolated tagmented DNA has a size distribution ranging from between about 250 bp to about 350 bp. In some embodiments, the isolated tagmented DNA has a size distribution ranging from between about 250 bp to about 320 bp. In some embodiments, the isolated tagmented DNA has a size distribution ranging from between about 250 bp to about 300 bp. In some embodiments, the method further comprises amplifying the isolated tagmented DNA to provide a plurality of amplicons. In some embodiments, the method further comprises sequencing the plurality of amplicons. In some embodiments, the sequencing of the amplicons comprises next generation sequencing.

An eighth aspect of the present disclosure are double stranded DNA fragments having a size ranging from between about 250 to about 350 bp, wherein the double stranded DNA fragments are prepared by: (i) obtaining a sample comprising double-stranded DNA; (ii) introducing a fragmentation composition comprising a histone-like protein and a transposition system to the obtained sample to provide a fragmentation reaction mixture; and (iii) heating the fragmentation reaction mixture for a predetermined amount of time at a predetermined temperature. In some embodiments, the histone-like protein is derived from a thermophilic or hyperthermopohilic Archaea. In some embodiments, the histone-like protein comprises an amino acid sequence having 85% identity to any one of SEQ ID NOS: 1 to 2. In some embodiments, the histone-like protein comprises an amino acid sequence having 90% identity to any one of SEQ ID NOS: 1 to 2. In some embodiments, the histone-like protein comprises an amino acid sequence having 95% identity to any one of SEQ ID NOS: 1 to 2. In some embodiments, the histone-like protein comprises any one of SEQ ID NOS: 1 to 2.

In some embodiments, a concentration of the histone-like protein in the fragmentation reaction mixture ranges from between about 2.5 ng/μL to about 25 ngμL. In some embodiments, a concentration of the histone-like protein in the fragmentation reaction mixture ranges from between about 5 ng/μL to about 20 ng/μL. In some embodiments, a concentration of the histone-like protein in the reaction mixture ranges from between about 7.5 ng/μL to about 20 ng/μL. In some embodiments, the transposition system comprises a transposase, a transposon, and adapters. In some embodiments, a concentration of the transposition system in the fragmentation reaction mixture ranges from between about 150 ng/μL to about 200 ng/μL.

In some embodiments, concentration of the double-stranded DNA in the composition ranges from between about 2 ng/μL to about 8 ng/μL. In some embodiments, a concentration of the double-stranded DNA in the composition ranges from between about 3 ng/μL to about 7 ng/μL. In some embodiments, a concentration of the double-stranded DNA in the composition is about 5 ng/μL. In some embodiments, a ratio of a concentration of the histone-like protein to a concentration of the double stranded DNA in the fragmentation reaction mixture ranges from between about 0.5:1 to about 5:1. In some embodiments, a ratio of a concentration of the histone-like protein to a concentration of the double stranded DNA in the fragmentation reaction mixture ranges from between about 1:1 to about 4:1.

In some embodiments, the preparation further comprises removing impurities from the reaction mixture. In some embodiments, the removing of the impurities comprises: (a) capturing the DNA fragments onto beads; (b) flushing impurities from the reaction mixture; and (c) eluting the captured DNA fragments from the beads.

A ninth aspect of the present disclosure is a fragmentation composition comprising one or more histone-like proteins, one or more transposition systems, and at least one additional component. In some embodiments, the histone-like protein comprises any one of SEQ ID NOS: 1 to 2. In some embodiments, the at least one additional component is selected from a buffer, a polyol, a salt, or DMSO.

A tenth aspect of the present disclosure is a kit comprising a fragmentation composition and a double stranded DNA sample.

An eleventh aspect of the present disclosure is a kit comprising a fragmentation composition and a polymerase.

A twelfth aspect of the present disclosure is a kit comprising a fragmentation composition and a next generation sequencing device.

A thirteenth aspect of the present disclosure is a kit comprising a fragmentation composition and one or more reagents for conducting a polymerase chain reaction.

A fourteenth aspect of the present disclosure is a kit comprising a fragmentation composition a solution comprising one or more divalent cations.

A fifteenth aspect of the present disclosure is a composition comprising a histone-like protein, a transposase, a transposon end composition, and one or more oligonucleotides. In some embodiments, the one or more oligonucleotides are synthetic oligonucleotides. In some embodiments, the one or more oligonucleotides are adapters. In some embodiments, the histone-like protein comprises any one of SEQ ID NOS: 1 to 2. In some embodiments, the composition further comprises a divalent cation. In some embodiments, the composition further comprises double stranded DNA.

A sixteenth aspect of the present disclosure is a method for processing a sample including genomic material comprising: (i) obtaining a tagmentation reaction mixture including comprising a transposition system; (ii) introducing double-stranded DNA and a histone-like protein to the tagmentation reaction mixture to provide a fragmentation reaction mixture; and (iii) heating the fragmentation reaction mixture to a predetermined temperature for a predetermined amount of time. In some embodiments, the double-stranded DNA and the histone-like protein are sequentially added to the tagmentation reaction mixture. In some embodiments, the double-stranded DNA and the histone-like protein are simultaneously added to the tagmentation reaction mixture. In some embodiments, the double-stranded DNA and the histone-like protein are first mixed together to form a DNA-histone-like protein solution, and then the DNA-histone-like protein solution is added to the tagmentation reaction mixture. In some embodiments, the histone-like protein is derived from a thermophilic or hyperthermopohilic Archaea. In some embodiments, the histone-like protein is an archaeal histone-like protein derived from one of a Thermococcus or a Pyrococcus. In some embodiments, the histone-like protein comprises an amino acid sequence having 85% identity to any one of SEQ ID NOS: 1 to 2. In some embodiments, the histone-like protein comprises an amino acid sequence having 90% identity to any one of SEQ ID NOS: 1 to 2. In some embodiments, the histone-like protein comprises an amino acid sequence having 95% identity to any one of SEQ ID NOS: 1 to 2. In some embodiments, the histone-like protein comprises any one of SEQ ID NOS: 1 to 2.

In some embodiments, concentration of the double-stranded DNA in the composition ranges from between about 2 ng/μL to about 8 ng/μL. In some embodiments, a concentration of the double-stranded DNA in the composition ranges from between about 3 ng/μL to about 7 ng/μL. In some embodiments, a concentration of the double-stranded DNA in the composition is about 5 ng/μL. In some embodiments, a ratio of a concentration of the histone-like protein in the fragmentation reaction mixture to a concentration of the double-stranded DNA in the tagmentation reaction mixture ranges from between about 1:1 to about 4:1. In some embodiments, the ratio ranges from between about 1.5:1 to about 3:1.

In some embodiments, the transposition system comprises a transposase, a transposon, and adapters. In some embodiments, the transposition system comprises a hyperactive Tn5 transposase and a Tn5-type transposase recognition site. In some embodiments, the transposition system comprises a hyperactive Tn5 transposase, a Tn5-type transposase recognition site, and one or more oligonucleotides. In some embodiments, the tagmentation reaction mixture further comprises a divalent cation is selected from the group consisting of Co²⁺, Mn²⁺, Mg²⁺, Cd²⁺, and Ca²⁺.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For a general understanding of the features of the disclosure, reference is made to the drawings. In the drawings, like reference numerals have been used throughout to identify identical elements.

FIG. 1 depicts a method of tagmenting DNA in the presence of one or more histone-like proteins in accordance with one embodiment of the present disclosure.

FIG. 2 illustrates a method of tagmenting DNA in the presence of one or more histone-like proteins in accordance with one embodiment of the present disclosure.

FIG. 3 illustrates a method of tagmenting DNA in the presence of one or more histone-like proteins in accordance with one embodiment of the present disclosure.

FIG. 4 depicts downstream processes which may be carried out following tagmentation of double stranded DNA.

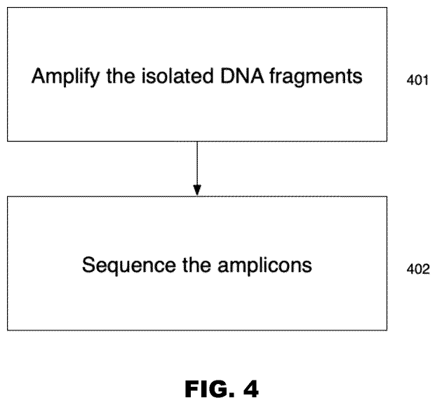

FIG. 5 illustrates the fragment size distributions of double stranded DNA after tagmentation using different concentrations of transposition system.

FIG. 6 illustrates the fragment size distributions of double stranded DNA after tagmentation in the presence of different concentrations of histone-like proteins.

FIG. 7 illustrates the insert sizes of tagmentation libraries prepared in the presence of a histone-like protein and a transposition system, where the concentration of histone-like protein was varied while the concentration of the transposition system was held constant.

FIG. 8 illustrates the insert sizes of tagmentation libraries prepared in the presence of different concentrations of a transposition system.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the term “amplicon refers to the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. In some embodiments, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references, each of which are incorporated herein by reference herein in their entirety: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g., “real-time PCR”, or “real-time NASBA” as described in Leone et al, Nucleic Acids Research, 26:2150-2155 (1998), and like references.

As used herein “amplification” refers to a process in which a copy number increases. Amplification may be a process in which replication occurs repeatedly over time to form multiple copies of a template. Amplification can produce an exponential or linear increase in the number of copies as amplification proceeds. Exemplary amplification strategies include polymerase chain reaction (PCR), loop-mediated isothermal amplification (LAMP), rolling circle replication (RCA), cascade-RCA, nucleic acid based amplification (NASBA), and the like. Also, amplification can utilize a linear or circular template. Amplification can be performed under any suitable temperature conditions, such as with thermal cycling or isothermally. Furthermore, amplification can be performed in an amplification mixture (or reagent mixture), which is any composition capable of amplifying a nucleic acid target, if any, in the mixture. PCR amplification relies on repeated cycles of heating and cooling (i.e., thermal cycling) to achieve successive rounds of replication. PCR can be performed by thermal cycling between two or more temperature setpoints, such as a higher denaturation temperature and a lower annealing/extension temperature, or among three or more temperature setpoints, such as a higher denaturation temperature, a lower annealing temperature, and an intermediate extension temperature, among others. PCR can be performed with a thermostable polymerase, such as Taq DNA polymerase. PCR generally produces an exponential increase in the amount of a product amplicon over successive cycles.

As used herein, the terms “barcode sequence” or “molecular barcode” refer to a unique sequence of nucleotides can be used to a) identify and/or track the source of a polynucleotide in a reaction, b) count how many times an initial molecule is sequenced and c) pair sequence reads from different strands of the same molecule. Barcode sequences may vary widely in size and composition; the following references provide guidance for selecting sets of barcode sequences appropriate for particular embodiments: Casbon (Nuc. Acids Res. 2011, 22 e81), Brenner, U.S. Pat. No. 5,635,400; Brenner et al, Proc. Natl. Acad. Sci., 97:1665-1670 (2000); Shoemaker et al, Nature Genetics, 14:450-456 (1996); Morris et al, European patent publication 0799897A1; Wallace, U.S. Pat. No. 5,981,179; and the like. In particular embodiments, a barcode sequence may have a length in range of from 2 to 36 nucleotides, or from 6 to 30 nucleotides, or from 8 to 20 nucleotides.

As used herein, the term “biological sample,” “tissue sample,” “specimen” or the like refers to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

As used herein, the term “fragment” refers to a portion of a larger polynucleotide molecule. A polynucleotide, for example, can be broken up, or fragmented into, a plurality of segments, either through natural processes, as is the case with, e.g., cfDNA fragments that can naturally occur within a biological sample, or through in vitro manipulation. A sample may be fragmented via tagmentation.

As used herein, the term “mixture” refers to a combination of elements, that are interspersed and not in any particular order. A mixture is heterogeneous and not spatially separable into its different constituents. Examples of mixtures of elements include a number of different elements that are dissolved in the same aqueous solution and a number of different elements attached to a solid support at random positions (i.e., in no particular order). A mixture is not addressable. To illustrate by example, an array of spatially separated surface-bound polynucleotides, as is commonly known in the art, is not a mixture of surface-bound polynucleotides because the species of surface-bound polynucleotides are spatially distinct and the array is addressable.

As used herein, the term “next generation sequencing” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Some examples of next generation sequencing techniques include, but are not limited to, sequencing by synthesis, sequencing by ligation, and sequencing by hybridization. These technologies produce shorter reads (anywhere from about 25-about 500 bp) but many hundreds of thousands or millions of reads in a relatively short time. Examples of such sequencing devices available from Illumina (San Diego, CA) include, but are not limited to iSEQ, MiniSEQ, MISEQ, NextSEQ, NoveSEQ. It is believed that the Illumina next-generation sequencing technology uses clonal amplification and sequencing by synthesis (SBS) chemistry to enable rapid sequencing. The process simultaneously identifies DNA bases while incorporating them into a nucleic acid chain. Each base emits a unique fluorescent signal as it is added to the growing strand, which is used to determine the order of the DNA sequence. A non-limiting example of a sequencing device available from ThermoFisher Scientific (Waltham, MA) includes the Ion Personal Genome Machine™ (PGM™) System. It is believed that Ion Torrent sequencing measures the direct release of H+ (protons) from the incorporation of individual bases by DNA polymerase. A non-limiting example of a sequencing device available from Pacific Biosciences (Menlo Park, CA) includes the PacBio Sequel Systems. A non-limiting example of a sequencing device available from Roche (Pleasanton, CA) is the Roche 454. Next-generation sequencing methods may also include nanopore sequencing methods. In general, three nanopore sequencing approaches have been pursued: strand sequencing in which the bases of DNA are identified as they pass sequentially through a nanopore, exonuclease-based nanopore sequencing in which nucleotides are enzymatically cleaved one-by-one from a DNA molecule and monitored as they are captured by and pass through the nanopore, and a nanopore sequencing by synthesis (SBS) approach in which identifiable polymer tags are attached to nucleotides and registered in nanopores during enzyme-catalyzed DNA synthesis. Common to all these methods is the need for precise control of the reaction rates so that each base is determined in order. Strand sequencing requires a method for slowing down the passage of the DNA through the nanopore and decoding a plurality of bases within the channel; ratcheting approaches, taking advantage of molecular motors, have been developed for this purpose. Exonuclease-based sequencing requires the release of each nucleotide close enough to the pore to guarantee its capture and its transit through the pore at a rate slow enough to obtain a valid ionic current signal. In addition, both of these methods rely on distinctions among the four natural bases, two relatively similar purines and two similar pyrimidines. The nanopore SBS approach utilizes synthetic polymer tags attached to the nucleotides that are designed specifically to produce unique and readily distinguishable ionic current blockade signatures for sequence determination. In some embodiments, sequencing of nucleic acids comprises via nanopore sequencing comprises preparing nanopore sequencing complexes and determining polynucleotide sequences. Methods of preparing nanopores and nanopore sequencing are described in U.S. Patent Application Publication No. 2017/0268052, and PCT Publication Nos. WO2014/074727, WO2006/028508, WO2012/083249, and WO/2014/074727, the disclosures of which are hereby incorporated by reference herein in their entireties. In some embodiments, tagged nucleotides may be used in the determination of the polynucleotide sequences (see, e.g., PCT Publication No. WO/2020/131759, WO/2013/191793, and WO/2015/148402, the disclosures of which are hereby incorporated by reference herein in their entireties). Analysis of the data generated by sequencing is generally performed using software and/or statistical algorithms that perform various data conversions, e.g., conversion of signal emissions into base calls, conversion of base calls into consensus sequences for a nucleic acid template, etc. Such software, statistical algorithms, and the use of such are described in detail, in U.S. Patent Application Publication Nos. 2009/0024331 2017/0044606 and in PCT Publication No. WO/2018/034745, the disclosures of which are hereby incorporated by reference herein in their entireties.

As used herein, the term “oligonucleotide” refers to a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers. An oligonucleotide may be 10 to 20, 11 to 30, 31 to 40, 41 to 50, 51-60, 61 to 70, 71 to 80, 80 to 100, 100 to 150 or 150 to 200 nucleotides in length, for example.

As used herein, the term “sequence,” when used in reference to a nucleic acid molecule, refers to the order of nucleotides (or bases) in the nucleic acid molecules. In cases, where different species of nucleotides are present in the nucleic acid molecule, the sequence includes an identification of the species of nucleotide (or base) at respective positions in the nucleic acid molecule. A sequence is a property of all or part of a nucleic acid molecule. The term can be used similarly to describe the order and positional identity of monomeric units in other polymers such as amino acid monomeric units of protein polymers.

As used herein, the term “sequencing” refers to the determination of the order and position of bases in a nucleic acid molecule. More particularly, the term “sequencing” refers to biochemical methods for determining the order of the nucleotide bases, adenine, guanine, cytosine, and thymine, in a DNA oligonucleotide. Sequencing, as the term is used herein, can include without limitation parallel sequencing or any other sequencing method known of those skilled in the art, for example, chain-termination methods, rapid DNA sequencing methods, wandering-spot analysis, Maxam-Gilbert sequencing, dye-terminator sequencing, or using any other modern automated DNA sequencing instruments.

As used herein, the terms “tagmentation” or “tagmenting” refer to the process in which genomic DNA is cleaved, tagged with adapter sequences, and extended to fill in gaps arising from the cleavage and tagging. More specifically, “tagmentation” refers to the modification of DNA by a transposome complex comprising transposase enzyme complexed with adaptors comprising transposon end sequence. Tagmentation results in the simultaneous fragmentation of the DNA and ligation of the adaptors to the 5′ ends of both strands of duplex fragments. Tagmentation results in the simultaneous fragmentation of the nucleic acid and ligation of the adaptors to the 5′ ends of both strands of duplex fragments. Following a purification step to remove the transposase enzyme, additional sequences can be added to the ends of the adapted fragments, for example by PCR, ligation, or any other suitable methodology known to those of skill in the art.

As used herein, the phrase “transposition reaction” refers to a reaction wherein one or more transposons are inserted into target nucleic acids at random sites or almost random sites. Essential components in a transposition reaction are a transposase and DNA oligonucleotides that exhibit the nucleotide sequences of a transposon, including the transferred transposon sequence and its complement (i.e., the non-transferred transposon end sequence) as well as other components needed to form a functional transposition or transposome complex. The DNA oligonucleotides can further comprise additional sequences (e.g., adaptor or primer sequences) as needed or desired.

### Overview

The present disclosure provides compositions and kits for the tagmentation of double stranded DNA. In some embodiments, the compositions and kits for the tagmentation of double stranded DNA include one or more histone-like proteins and/or one or more transposition systems. The present disclosure also provides methods for the tagmentation of double stranded DNA in the presence of one or more histone-like proteins. Following tagmentation of the double stranded DNA in the presence of the one or more histone-like proteins, the tagmented DNA may then be amplified and/or sequenced.

### Compositions

In one aspect of the present disclosure are compositions for use in a tagmentation reaction. In some embodiments, the compositions include one or more histone-like proteins and at least one additional component.

In some embodiments, the present disclosure provides a fragmentation composition comprising one or more histone-like proteins and one or more transposition systems. In some embodiments, a fragmentation composition comprises one or more histone-like proteins, one or more transposition systems, and one or more additional components, e.g., a divalent cation, a buffer, a polyol, DMSO, etc.

In some embodiments, the present disclosure provides a fragmentation reaction mixture comprising one or more histone-like proteins, one or more transposition systems, and double stranded DNA. In some embodiments, the present disclosure provides a fragmentation reaction mixture comprising one or more histone-like proteins, one or more transposition systems, double stranded DNA, and one or more additional components, e.g., a divalent cation, a buffer, a polyol, DMSO, etc.

In some embodiments, the present disclosure also provides for a tagmentation reaction mixture comprising one or more transposition systems and one or more optional additional components, e.g., a divalent cation, a buffer, a polyol, DMSO, etc.

In some embodiments, the present disclosure provides for DNA-histone-like protein solutions comprising one or more histone-like proteins and double stranded DNA.

Each of the components of the fragmentation composition, the fragmentation reaction mixture, and the tagmentation reaction mixture are described herein.

### Histone-Like Proteins

As noted above, the disclosed fragmentation compositions and fragmentation reaction mixtures each include one or more histone-like proteins. Histone-like proteins (HLPs) are small and basic bacterial proteins that are associated with a nucleoid and play roles in maintaining DNA architecture and regulating DNA transactions such as replication, recombination/repair and transcription. Architectural chromatin proteins are found in every domain of life. In eukaryotes and most archaeal lineages, histones are responsible for packaging and compaction of the DNA. Archaeal histone-like proteins exhibit some homology to eukaryal core histones in primary sequence, secondary and tertiary structures.

It is believed that the histone-like proteins form homo- and/or heterodimers and act in wrapping DNA by forming a tetramer which consists of a dimer of dimers to form what is termed a nucleosome-like structure. The histone-like proteins are present as dimers in solutions without DNA and form stable tetramers in solution containing double stranded DNA. These proteins have been shown to compact DNA by forming these nucleosome-like structures which wrap DNA around the protein with a footprint of about 90 bp (for HphA homotetramer). When these histone-like proteins form nucleosome-like structures, it is believed that they provide a degree of nuclease protection by sterically hindering a nuclease or blocking the recognition sites of a nuclease.

In some embodiments, the histone-like protein is an archaeal histone-like protein. In other embodiments, the histone-like protein is derived from a thermophilic or hyperthermopohilic Archaea. In some embodiments, the archaeal histone-like protein is derived from a Thermococcus. In other embodiments, the archaeal histone-like protein is derived from a Pyrococcus. In other embodiments, the archaeal histone-like protein is derived from Methanobacterium thermoautotrophicum. In other embodiments, the archaeal histone-like protein is derived from Methanothermus fervidus. In other embodiments, the archaeal histone-like protein is derived from a Pyrococcus horikoshii. In other embodiments, the archaeal histone-like protein is derived from a Pyrococcus horikoshii OT3.

Yet other archaeal histone-like proteins may be derived from Thaumarchaeota, Aigarchaeota, Crenarchaeota, Korarchaeota (TACK), Diapherotrites, Pacearchaeota, Aenigmarchaeota, Nanoarchaeota, Nanohaloarchaeota (DPANN), and Asgard Archaea. Yet further archaeal histone-like proteins may be derived from Asgard Archaea and candidate phyla Bathyarchaeota, Woesearchaeota, Pacearchaeota, Aenigmarchaeota, Diapherotrites, Huberarchaea, and Micrarchaeota. Even further archaeal histone-like proteins are described by Henneman et al.,” Structure and function of archaeal histones,” PLOS Genetics | https://doi.org/10.1371/journal.pgen.1007582 Sep. 13, 2018, the disclosure of which is hereby incorporated by reference herein in its entirety.

In some embodiments, an amino acid sequence encoding the histone-like protein has at least 80% sequence identity to any one of SEQ ID NOS: 1 and 2. In some embodiments, an amino acid sequence encoding the histone-like protein has at least 85% sequence identity to any one of SEQ ID NOS: 1 and 2. In some embodiments, an amino acid sequence encoding the histone-like protein has at least 86% sequence identity to any one of SEQ ID NOS: 1 and 2. In some embodiments, an amino acid sequence encoding the histone-like protein has at least 87% sequence identity to any one of SEQ ID NOS: 1 and 2. In some embodiments, an amino acid sequence encoding the histone-like protein has at least 88% sequence identity to any one of SEQ ID NOS: 1 and 2. In some embodiments, an amino acid sequence encoding the histone-like protein has at least 89% sequence identity to any one of SEQ ID NOS: 1 and 2. In some embodiments, an amino acid sequence encoding the histone-like protein has at least 90% sequence identity to any one of SEQ ID NOS: 1 and 2. In some embodiments, an amino acid sequence encoding the histone-like protein has at least 91% sequence identity to any one of SEQ ID NOS: 1 and 2. In some embodiments, an amino acid sequence encoding the histone-like protein has at least 92% sequence identity to any one of SEQ ID NOS: 1 and 2. In some embodiments, an amino acid sequence encoding the histone-like protein has at least 93% sequence identity to any one of SEQ ID NOS: 1 and 2. In some embodiments, an amino acid sequence encoding the histone-like protein has at least 94% sequence identity to any one of SEQ ID NOS: 1 and 2. In some embodiments, an amino acid sequence encoding the histone-like protein has at least 95% sequence identity to any one of SEQ ID NOS: 1 and 2. In some embodiments, an amino acid sequence encoding the histone-like protein has at least 96% sequence identity to any one of SEQ ID NOS: 1 and 2. In some embodiments, an amino acid sequence encoding the histone-like protein has at least 97% sequence identity to any one of SEQ ID NOS: 1 and 2. In some embodiments, an amino acid sequence encoding the histone-like protein has at least 98% sequence identity to any one of SEQ ID NOS: 1 and 2. In some embodiments, an amino acid sequence encoding the histone-like protein has at least 99% sequence identity to any one of SEQ ID NOS: 1 and 2. In some embodiments, an amino acid sequence encoding the histone-like protein has any one of SEQ ID NOS: 1 and 2.

In some embodiments, the histone-like protein has at least 80% sequence identity to SEQ ID NO: 3. In some embodiments, the histone-like protein has at least 85% sequence identity to SEQ ID NO: 3. In some embodiments, the histone-like protein has at least 86% sequence identity to SEQ ID NO: 3. In some embodiments, the histone-like protein has at least 87% sequence identity to SEQ ID NO: 3. In some embodiments, the histone-like protein has at least 88% sequence identity to SEQ ID NO: 3. In some embodiments, the histone-like protein has at least 89% sequence identity to SEQ ID NO: 3. In some embodiments, the histone-like protein has at least 90% sequence identity to SEQ ID NO: 3. In some embodiments, the histone-like protein has at least 91% sequence identity to SEQ ID NO: 3. In some embodiments, the histone-like protein has at least 92% sequence identity to SEQ ID NO: 3. In some embodiments, the histone-like protein has at least 93% sequence identity to SEQ ID NO: 3. In some embodiments, the histone-like protein has at least 94% sequence identity to SEQ ID NO: 3. In some embodiments, the histone-like protein has at least 95% sequence identity to SEQ ID NO: 3. In some embodiments, the histone-like protein has at least 96% sequence identity to SEQ ID NO: 3. In some embodiments, the histone-like protein has at least 97% sequence identity to SEQ ID NO: 3. In some embodiments, the histone-like protein has at least 98% sequence identity to SEQ ID NO: 3. In some embodiments, the histone-like protein has at least 99% sequence identity to SEQ ID NO: 3. In some embodiments, the histone-like protein has SEQ ID NO: 3.

In some embodiments, the histone-like protein has at least 80% sequence identity to SEQ ID NO: 4. In some embodiments, the histone-like protein has at least 85% sequence identity to SEQ ID NO: 4. In some embodiments, the histone-like protein has at least 86% sequence identity to SEQ ID NO: 4. In some embodiments, the histone-like protein has at least 87% sequence identity to SEQ ID NO: 4. In some embodiments, the histone-like protein has at least 88% sequence identity to SEQ ID NO: 4. In some embodiments, the histone-like protein has at least 89% sequence identity to SEQ ID NO: 4. In some embodiments, the histone-like protein has at least 90% sequence identity to SEQ ID NO: 4. In some embodiments, the histone-like protein has at least 91% sequence identity to SEQ ID NO: 4. In some embodiments, the histone-like protein has at least 92% sequence identity to SEQ ID NO: 4. In some embodiments, the histone-like protein has at least 93% sequence identity to SEQ ID NO: 4. In some embodiments, the histone-like protein has at least 94% sequence identity to SEQ ID NO: 4. In some embodiments, the histone-like protein has at least 95% sequence identity to SEQ ID NO: 4. In some embodiments, the histone-like protein has at least 96% sequence identity to SEQ ID NO: 4. In some embodiments, the histone-like protein has at least 97% sequence identity to SEQ ID NO: 4. In some embodiments, the histone-like protein has at least 98% sequence identity to SEQ ID NO: 4. In some embodiments, the histone-like protein has at least 99% sequence identity to SEQ ID NO: 4. In some embodiments, the histone-like protein has SEQ ID NO: 4.

In some embodiments, a concentration of the histone-like protein in any composition or reaction mixture ranges from between about 1.5 ng/μL to about 50 ng/μL. In other embodiments, a concentration of the histone-like protein in any composition or reaction mixture ranges from between about 2 ng/μL to about 40 ng/μL. In other embodiments, a concentration of the histone-like protein in any composition or reaction mixture ranges from between about 2 ng/μL to about 35 ng/μL. In other embodiments, a concentration of the histone-like protein in any composition or reaction mixture ranges from between about 2.5 ng/μL to about 35 ng/μL. In other embodiments, a concentration of the histone-like protein in any composition or reaction mixture ranges from between about 2.5 ng/μL to about 30 ng/μL. In other embodiments, a concentration of the histone-like protein in any composition or reaction mixture ranges from between about 2.5 ng/μL to about 25 ng/μL. In other embodiments, a concentration of the histone-like protein in any composition or reaction mixture ranges from between about 2.5 ng/μL to about 20 ng/μL. In other embodiments, a concentration of the histone-like protein in any composition or reaction mixture ranges from between about 3 ng/μL to about 30 ng/μL. In other embodiments, a concentration of the histone-like protein in any composition or reaction mixture ranges from between about 3 ng/μL to about 25 ng/μL. In other embodiments, a concentration of the histone-like protein in any composition or reaction mixture ranges from between about 3 ng/μL to about 20 ng/μL. In other embodiments, a concentration of the histone-like protein in any composition or reaction mixture ranges from between about 4 ng/μL to about 35 ng/μL. In other embodiments, a concentration of the histone-like protein in any composition or reaction mixture ranges from between about 4 ng/μL to about 30 ng/μL. In other embodiments, a concentration of the histone-like protein in any composition or reaction mixture ranges from between about 4 ng/μL to about 25 ng/μL. In other embodiments, a concentration of the histone-like protein in any composition or reaction mixture ranges from between about 4 ng/μL to about 20 ng/μL. In other embodiments, a concentration of the histone-like protein in any composition or reaction mixture ranges from between about 5 ng/μL to about 35 ng/μL. In other embodiments, a concentration of the histone-like protein in any composition or reaction mixture ranges from between about 5 ng/μL to about 30 ng/μL. In other embodiments, a concentration of the histone-like protein in any composition or reaction mixture ranges from between about 5 ng/μL to about 25 ng/μL. In other embodiments, a concentration of the histone-like protein in any composition or reaction mixture ranges from between about 5 ng/μL to about 15 ng/μL. In other embodiments, a concentration of the histone-like protein in any composition or reaction mixture ranges from between about 6 ng/μL to about 30 ng/μL. In other embodiments, a concentration of the histone-like protein in any composition or reaction mixture ranges from between about 6 ng/μL to about 25 ng/μL. In other embodiments, a concentration of the histone-like protein in any composition or reaction mixture ranges from between about 6 ng/μL to about 20 ng/μL. In other embodiments, a concentration of the histone-like protein in any composition or reaction mixture ranges from between about 6 ng/μL to about 15 ng/μL. In other embodiments, a concentration of the histone-like protein in any composition or reaction mixture ranges from between about 7 ng/μL to about 30 ng/μL. In other embodiments, a concentration of the histone-like protein in any composition or reaction mixture ranges from between about 7 ng/μL to about 25 ng/μL. In other embodiments, a concentration of the histone-like protein in any composition or reaction mixture ranges from between about 7 ng/μL to about 20 ng/μL. In other embodiments, a concentration of the histone-like protein in any composition or reaction mixture ranges from between about 7 ng/μL to about 15 ng/μL. In other embodiments, a concentration of the histone-like protein in any composition or reaction mixture ranges from between about 8 ng/μL to about 25 ng/μL. In other embodiments, a concentration of the histone-like protein in any composition or reaction mixture ranges from between about 8 ng/μL to about 20 ng/μL. In other embodiments, a concentration of the histone-like protein in any composition or reaction mixture ranges from between about 8 ng/μL to about 15 ng/μL.

In some embodiments, the histone-like protein is derived from a Pyrococcus horikoshii OT3, and wherein a concentration of the histone-like protein in any composition or reaction mixture is about 5 ng/μL. In some embodiments, the histone-like protein is derived from a Pyrococcus horikoshii OT3, and wherein a concentration of the histone-like protein in any composition or reaction mixture is about 7.5 ng/μL. In some embodiments, the histone-like protein is derived from a Pyrococcus horikoshii OT3, and wherein a concentration of the histone-like protein in any composition or reaction mixture is about 10 ng/μL. In some embodiments, the histone-like protein is derived from a Pyrococcus horikoshii OT3, and wherein a concentration of the histone-like protein in any composition or reaction mixture is about 15 ng/μL. In some embodiments, the histone-like protein is derived from a Pyrococcus horikoshii OT3, and wherein a concentration of the histone-like protein in any composition or reaction mixture is about 20 ng/μL.

In some embodiments, the histone-like protein has at least 85% identity to any one of SEQ ID NOS: 1 and 2, and wherein a concentration of the histone-like protein in any composition or reaction mixture is about 5 ng/μL. In some embodiments, the histone-like protein has at least 85% identity to any one of SEQ ID NOS: 1 and 2, and wherein a concentration of the histone-like protein in any composition or reaction mixture is about 7.5 ng/μL. In some embodiments, the histone-like protein has at least 85% identity to any one of SEQ ID NOS: 1 and 2, and wherein a concentration of the histone-like protein in any composition or reaction mixture is about 10 ng/μL. In some embodiments, the histone-like protein has at least 85% identity to any one of SEQ ID NOS: 1 and 2, and wherein a concentration of the histone-like protein in any composition or reaction mixture is about 15 ng/μL. In some embodiments, the histone-like protein has at least 85% identity to any one of SEQ ID NOS: 1 and 2, and wherein a concentration of the histone-like protein in any composition or reaction mixture is about 20 ng/μL.

### Double Stranded DNA

In some embodiments, the compositions and/or reaction mixtures of the present disclosure include double stranded DNA. In some embodiments, the double stranded DNA is cDNA, ctDNA, or cfDNA. Of course, the skilled artisan will appreciate that any DNA suitable for use in the present disclosure may have first been converted from RNA using techniques known in the art.

The double stranded DNA may be obtained from any source. For example, double stranded DNA may be obtained from a single organism or from populations of nucleic acid molecules obtained from natural sources that include one or more organisms. Sources of nucleic acid molecules include, but are not limited to, organelles, cells, tissues, organs, organisms, single cell, or a single organelle. Cells that may be used as sources of target nucleic acid molecules may be prokaryotic (bacterial cells, for example, Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces genera); archeaon, such as crenarchaeota, nanoarchaeota or euryarchaeotia; or eukaryotic such as fungi, (for example, yeasts), plants, protozoans and other parasites, and animals (including insects (for example, Drosophila spp.), nematodes (e.g., Caenorhabditis elegans), and mammals (for example, rat, mouse, monkey, non-human primate and human).

In some embodiments, the double stranded DNA is genomic DNA derived from a mammalian subject, e.g., a human patient. In some embodiments, the double stranded DNA is derived from a tumor sample, e.g., from a tumor sample derived from a human patient.

In some embodiments, the double stranded DNA can be enriched for certain sequences of interest prior to tagmentation. U.S. Pat. Nos. 10,590,471, 10,900,068, 10,907,204 and 9,365,897; United States Patent Publication Nos. 2020/0048694 and 2020/0392483; and PCT Publication WO/2012/108864 describe various methods of enriching for sequences of interest, the disclosures of which are hereby incorporated by reference herein in their entireties.

In some embodiments, a concentration of the double-stranded DNA in any DNA-histone-protein like solution or fragmentation reaction mixture ranges from between about 0.5 ng/μL to about 15 ng/μL. In some embodiments, a concentration of the double-stranded DNA in any DNA-histone-protein like solution or fragmentation reaction mixture ranges from between about 1 ng/μL to about 10 ng/μL. In some embodiments, a concentration of the double-stranded DNA in any DNA-histone-protein like solution or fragmentation reaction mixture ranges from between about 2 ng/μL to about 8 ng/μL. In some embodiments, a concentration of the double-stranded DNA in any DNA-histone-protein like solution or fragmentation reaction mixture ranges from between about 3 ng/μL to about 7 ng/μL. In some embodiments, a concentration of the double-stranded DNA in any DNA-histone-protein like solution or fragmentation reaction mixture ranges from between about 4 ng/μL to about 6 ng/μL. In some embodiments, a concentration of the double-stranded DNA in any DNA-histone-protein like solution or fragmentation reaction mixture is about 4 ng/μL, about 5 ng/μL, about 6 ng/μL, or about 7 ng/μL.

In some embodiments, a ratio of a concentration of double stranded DNA to a concentration of histone-like protein in any DNA-histone-protein like solution or fragmentation reaction mixture ranges from between about 0.25:1 to about 6:1. In some embodiments, a ratio of a concentration of double stranded DNA to a concentration of histone-like protein in any DNA-histone-protein like solution or fragmentation reaction mixture ranges from between about 0.5:1 to about 5:1. In some embodiments, a ratio of a concentration of double stranded DNA to a concentration of histone-like protein in any DNA-histone-protein like solution or fragmentation reaction mixture ranges from between about 1:1 to about 4:1. In some embodiments, a ratio of a concentration of double stranded DNA to a concentration of histone-like protein in any DNA-histone-protein like solution or fragmentation reaction mixture ranges from between about 1.5:1 to about 3:1. In some embodiments, a ratio of a concentration of double stranded DNA to a concentration of histone-like protein in any DNA-histone-protein like solution or fragmentation reaction mixture ranges from between about 2:1 to about 1:2.

### Transposition System

As noted above, the fragmentation compositions and the tagmentation reaction mixtures of the present disclosure each include one or more transposition systems. In some embodiments, any transposition system may be utilized in the fragmentation compositions or the tagmentation reaction mixtures provided that the transposition system is capable of fragmenting DNA. Suitable transpositions systems are described by Adey et. al., “Rapid, low-input, low-bias construction of shotgun fragment libraries by high-density in vitro transposition,” Genome Biology 2010, 11:R119, the disclosure of which is hereby incorporated by reference herein in its entirety.

In some embodiments, the transposition system includes a transposase, a transposon or transposon DNA, and one or more oligonucleotides (e.g., barcodes, tags, adapters, etc.). In some embodiments, the transposase is complexed with a transposon DNA including a double stranded transposase binding site and a first nucleic acid sequence including one or more of a tag, an adapter, or a barcode sequence and a priming site to form a transposase/transposon DNA complex. In some embodiments, the first nucleic acid sequence may be in the form of a single stranded extension or the first nucleic acid sequence may be in the form of a loop with each end connected to a corresponding strand of the double stranded transposase binding site. In some embodiments, the transposases have the capability to bind to the transposon DNA and dimerize when contacted together forming a transposase/transposon DNA complex dimer called transposome. In some embodiments, the transposomes have the capability to bind to target locations along double stranded nucleic acids forming a complex including the transposome and the double stranded genomic DNA. Such transposition systems are described in U.S. Pat. No. 10,894,980, in United States Patent Publication No. 2018/0305683, and in PCT Publication No. WO/2015/089339, the disclosures of which are hereby incorporated by reference herein in their entireties.

In some embodiments, the transposase complexed with the transposon DNA comprises a dimer of a transposase and a pair of adapters (see United States Patent Publication No. 2018/0305683, the disclosure of which is hereby incorporated by reference herein in its entirety). The term “adaptor” refers to a nucleic acid that can be joined, via a transposase-mediated reaction, to at least one strand of a double-stranded nucleic acid molecule (e.g., double stranded DNA). The adapters may be at least partially double-stranded and be 30 to 150 bases in length, e.g., 40 to 120 bases. In other embodiments, the transposase complex comprises a transposase loaded with two adaptor molecules that each include a recognition sequence for the transposase at one end. In yet other embodiments, the transposase complexed with the transposon DNA comprises a dimer of modified transposase Tn5 and a pair of Tn-5 binding double stranded DNA oligonucleotides containing a 19 base pair transposase-binding sequence (mosaic end) or inverted repeat sequence. In even further embodiments, the transposition comprises at least one first oligonucleotide comprising at least one double-stranded portion, wherein the double-stranded portion comprises at least one first recognition end sequence; at least one second oligonucleotide comprising at least one double-stranded portion, wherein the double-stranded portion comprises at least one second recognition end sequence; and a transposase (see PCT Publication No. WO/2015/089339, the disclosure of which is hereby incorporated by reference herein in its entirety).

In other embodiments, the transposition system includes a transposase, a transposon end composition, and/or adapters. Here, the term “transposase” refers to an enzyme that is capable of forming a functional complex with a transposon end-containing composition (e.g., transposons, transposon ends, transposon end compositions) and catalyzing insertion or transposition of the transposon end-containing composition into the double-stranded target DNA with which it is incubated in an in vitro transposition reaction. As used herein, the phrase “transposon end composition” refers to a composition comprising a transposon end (i.e., the minimum double-stranded DNA segment that is capable of acting with a transposase to undergo a transposition reaction), optionally plus additional sequence or sequences. For example, a transposon end attached to a tag is a “transposon end composition.” In some embodiments, the transposon end composition includes two transposon end oligonucleotides including the “transferred transposon end oligonucleotide” or “transferred strand” and the “non-transferred strand end oligonucleotide,” or “non-transferred strand” which, in combination, exhibit the sequences of the transposon end, and in which one or both strand comprise additional sequence. Such transposition systems are described in U.S. Pat. Nos. 11,118,175, 10,815,478, and 10,184,122, the disclosures of which are hereby incorporated by reference herein in their entireties.

In some embodiments, the transposition system comprises TnAa. In some embodiments, the transposition system may utilize a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35:785, 1983; Savilahti, H, et al., EMBO J., 14:4893, 1995). In some other, the transposition system may utilize Staphylococcus aureus Tn552 (Colegio et al., J. Bacteriol., 183:2384-8, 2001; Kirby C et al., Mol. Microbiol., 43:173-86, 2002), Ty1 (Devine & Boeke, Nucleic Acids Res., 22:3765-72, 1994 and International Publication WO 95/23875), Transposon Tn7 (Craig, N L, Science. 271:1512, 1996; Craig, N L, Review in: Curr Top Microbiol Immunol., 204:27-48, 1996), Tn/O and IS10 (Kleckner N, et al., Curr Top Microbiol Immunol., 204:49-82, 1996), Mariner transposase (Lampe D J, et al., EMBO J., 15:5470-9, 1996), Tcl (Plasterk R H, Curr. Topics Microbiol. Immunol., 204:125-43, 1996), P Element (Gloor, G B, Methods Mol. Biol., 260:97-114, 2004), Tn3 (Ichikawa & Ohtsubo, J Biol. Chem. 265:18829-32, 1990), bacterial insertion sequences (Ohtsubo & Sekine, Curr. Top. Microbiol. Immunol. 204:1-26, 1996), retroviruses (Brown, et al., Proc Natl Acad Sci USA, 86:2525-9, 1989), and retrotransposon of yeast (Boeke & Corces, Annu Rev Microbiol. 43:403-34, 1989). Yet further transposition systems may include ISS, Tn10, Tn903, IS911, and engineered versions of transposase family enzymes (Zhang et al., (2009) PLOS Genet. 5: e1000689. Epub 2009 Oct. 16; Wilson C. et al (2007) J. Microbiol. Methods 71:332-5).

Even further useful transposon systems include the Tn3 transposon system (see Maekawa, T., Yanagihara, K., and Ohtsubo, E. (1996), A cell-free system of Tn3 transposition and transposition immunity, Genes Cells 1, 1007-1016), the Tn10 tranposon system (see Chalmers, R., Sewitz, S., Lipkow, K., and Crellin, P. (2000), Complete nucleotide sequence of Tn10, J. Bacteriol 182, 2970-2972), Piggybac transposon system (see Li, X., Burnight, E. R., Cooney, A. L, Malani, N., Brady, T., Sander, J. D., Staber, J., Wheelan, S. J., Joung, J. K., McCray, P. B., Jr., et al. (2013), PiggyBac transposase tools for genome engineering, Proc. Natl. Acad. Sci. USA 110, E2279-2287), Sleeping beauty transposon system (see Ivies, Z., Hackett, P. B., Piasterk, R. H., and Izsvak, Z. (1997), Molecular reconstruction of Sleeping Beauty, theTc1-like transposon from fish, and its transposition in human cells, Cell 91, 501-510), the Tol2 transposon system (see Kawakami, K. (2007), Tol2: a versatile gene transfer vector in vertebrates, Genome Biol. 8 Suppl. 1, S7.). Yet other suitable transposition systems include, for example, those provided by Illumina in the NEXTERA DNA or NEXTERA DNA Flex library preparation kit. Yet even further transposition systems or components thereof are described in United States U.S. Pat. Nos. 7,608,434, 7,083,980, 5,965,443, and 5,925,545, the disclosures of which are hereby incorporated by reference herein in their entireties.

In some embodiments, the transposase included within any transposition system has 95% sequence identity to SEQ ID NO: 5 (TnAa). In some embodiments, the transposase has 96% sequence identity to SEQ ID NO: 5. In some embodiments, the transposase has 97% sequence identity to SEQ ID NO: 5. In some embodiments, the transposase has 98% sequence identity to SEQ ID NO: 5. In some embodiments, the transposase has 99% sequence identity to SEQ ID NO: 5. In some embodiments, the transposase has SEQ ID NO: 5. In some embodiments, the transposase has at least 95% sequence identity to SEQ ID NO: 6. In some embodiments, the transposase has at least 97% sequence identity to SEQ ID NO: 6. In some embodiments, the transposase has at least 99% sequence identity to SEQ ID NO: 6. In some embodiments, the transposase has SEQ ID NO: 6.

In some embodiments, a concentration of the transposition system in any composition or reaction mixture ranges from between about 100 ng/μL to about 400 ng/μL. In some embodiments, a concentration of the transposition system in any composition or reaction mixture ranges from between about 100 ng/μL to about 250 ng/μL. In other embodiments, a concentration of the transposition system in any composition or reaction mixture ranges from between about 120 ng/μL to about 230 ng/μL. In other embodiments, a concentration of the transposition system in any composition or reaction mixture ranges from between about 140 ng/μL to about 210 ng/μL. In other embodiments, a concentration of the transposition system in any composition or reaction mixture ranges from between about 150 ng/μL to about 200 ng/μL. In other embodiments, a concentration of the transposition system in any composition or reaction mixture ranges from between about 160 ng/μL to about 190 ng/μL. In other embodiments, a concentration of the transposition system in any composition or reaction mixture is about 160 ng/μL. In other embodiments, a concentration of the transposition system in any composition or reaction mixture is about 170 ng/μL. In other embodiments, a concentration of the transposition system in any composition or reaction mixture is about 175 ng/μL. In other embodiments, a concentration of the transposition system in any composition or reaction mixture is about 180 ng/μL. In other embodiments, a concentration of the transposition system in any composition or reaction mixture is about 185 ng/μL. In other embodiments, a concentration of the transposition system in any composition or reaction mixture is about 190 ng/μL. In other embodiments, a concentration of the transposition system in any composition or reaction mixture is about 200 ng/μL.

### Divalent Cation

In some embodiments, the fragmentation composition, the fragmentation reaction mixture, and/or the tagmentation reaction mixture may include one or more divalent cations. In some embodiments, the divalent cation is selected from Co²⁺, Mn²⁺, Mg²⁺, Cd²⁺, and Ca²⁺.

In some embodiments, any of the compositions or reaction mixtures of the present disclosure may include a concentration of divalent cation which is at least about 0.01 mM, 0.02 mM, 0.05 mM, 0.1 mM, 0.2 mM, 0.5 mM, 1 mM, 2 mM, 5 mM, 8 mM, 10 mM, 12 mM, 15 mM, 20 mM, 30 mM, 40 mM, 50 mM, 60, mM, 70, mM, 80, mM, 90 mM, 100 mM. In some embodiments, any of the compositions or reaction mixtures of the present disclosure may have a concentration of a CoCl₂ of at least about 0.01 mM, 0.02 mM, 0.05 mM, 0.1 mM, 0.2 mM, 0.5 mM, 1 mM, 2 mM, 5 mM, 8 mM, 10 mM, 12 mM, 15 mM, 20 mM, 30 mM, 40 mM, 50 mM, 60, mM, 70, mM, 80, mM, 90 mM, 100 mM. In some embodiments, any of the compositions or reaction mixtures of the present disclosure may have a concentration of MnCl₂ which is at least about 0.01 mM, 0.02 mM, 0.05 mM, 0.1 mM, 0.2 mM, 0.5 mM, 1 mM, 2 mM, 5 mM, 8 mM, 10 mM, 12 mM, 15 mM, 20 mM, 30 mM, 40 mM, 50 mM, 60, mM, 70, mM, 80, mM, 90 mM, 100 mM. In some embodiments, any of the compositions or reaction mixtures of the present disclosure may have a concentration of MgCl₂ which is at least about 0.01 mM, 0.02 mM, 0.05 mM, 0.1 mM, 0.2 mM, 0.5 mM, 1 mM, 2 mM, 5 mM, 8 mM, 10 mM, 12 mM, 15 mM, 20 mM, 30 mM, 40 mM, 50 mM, 60, mM, 70, mM, 80, mM, 90 mM, 100 mM. In some embodiments, any of the compositions or reaction mixtures of the present disclosure may have a concentration of CdCl₂ which is at least about 0.01 mM, 0.02 mM, 0.05 mM, 0.1 mM, 0.2 mM, 0.5 mM, 1 mM, 2 mM, 5 mM, 8 mM, 10 mM, 12 mM, 15 mM, 20 mM, 30 mM, 40 mM, 50 mM, 60, mM, 70, mM, 80, mM, 90 mM, 100 mM. In some embodiments, any of the compositions or reaction mixtures of the present disclosure may have a concentration of CaCl₂) which is at least about 0.01 mM, 0.02 mM, 0.05 mM, 0.1 mM, 0.2 mM, 0.5 mM, 1 mM, 2 mM, 5 mM, 8 mM, 10 mM, 12 mM, 15 mM, 20 mM, 30 mM, 40 mM, 50 mM, 60, mM, 70, mM, 80, mM, 90 mM, 100 mM.

### Buffers

In some embodiments, any of the compositions or reaction mixtures of the present disclosure include one or more buffers. Non-liming examples of buffers include citric acid, potassium dihydrogen phosphate, boric acid, diethyl barbituric acid, piperazine-N,N′-bis(2-ethanesulfonic acid) (PIPES), dimethylarsinic acid, 2-(N-morpholino) ethanesulfonic acid, tris(hydroxymethyl)methylamine (TRIS), 2-(N-morpholino) ethanesulfonic acid (TAPS), N,N-bis(2-hydroxyethyl)glycine (Bicine), N-tris(hydroxymethyl)methylglycine (Tricine), 4-2-hydroxyethyl-1-piperazineethanesulfonic acid (HEPES), 2-{[tris(hydroxymethyl)methyl]amino}ethanesulfonic acid (TES), and combinations thereof. In other embodiments, the buffer may be comprised of tris(hydroxymethyl)methylamine (TRIS), 2-(N-morpholino)ethanesulfonic acid (TAPS), N,N-bis(2-hydroxyethyl)glycine (Bicine), N-tris(hydroxymethyl)methylglycine (Tricine), 4-2-hydroxyethyl-1-piperazineethanesulfonic acid (HEPES), 2-{[tris(hydroxymethyl)methyl]amino}ethanesulfonic acid (TES), or a combination thereof.

### Additional Components

In some embodiments, any of the compositions or reaction mixtures of the present disclosure include a polyol. Suitable polyols include 1,2-ethanediol, 1,2-propanediol, 1,3-propanediol, 2-methyl-1,3-propanediol, 2-methyl-1,2-propanediol, 2,2-dimethyl-1,3-propanediol, 2,2-diethyl-1,3-propanediol, 2-methyl-2-propyl-1,3-propanediol, 2-butyl-2-ethyl-1,3-propanediol, dihydroxyacetone, 2,2-dibutyl-1,3-propanediol, 3-methoxy-1,3-propanediol, 3-methoxy-1,2-propanediol, 3-methoxy-2,3-propanediol, 2-methoxymethyl-1,3-propanediol, 3-ethoxy-1,3-propanediol, 3-ethoxy-1,2-propanediol, 3-ethoxy-2,3-propanediol, 3-allyloxy-1,2-propanediol, 1,2-butanediol, 1,3-butanediol, 1,4-butanediol, 2,3-butanediol, 2,3-dimethyl-2,3-butanediol, 3,3-dimethyl-1,2-butanediol, 1,2-pentanediol, 1,3-pentanediol, 1,4-pentanediol, 1,5-pentanediol, 2,3-pentanediol, 2,4-pentanediol, 2-methyl-2,4-pentanediol, 2,4-dimethyl-2,4-pentanediol, 2,2,4-trimethyl-1,3-pentanediol, 1,2-hexanediol, 1,3-hexanediol, 1,4-hexanediol, 1,5-hexanediol, 1,6-hexanediol, 2,3-hexanediol, 2,4-hexanediol, 2,5-hexanediol, 3,4-hexanediol, 2,5-dimethyl-2,5-hexanediol, 2-ethyl-1,3-hexanediol, 1,2-heptanediol, 1,3-heptanediol, 1,4-heptanediol, 1,5-heptanediol, 1,6-heptanediol, 1,7-heptanediol, 1,8-octanediol, 1,2-octanediol, 1,3-octanediol 1,4-octanediol, 1,5-octanediol, 1,6-octanediol, 1,7-octanediol, 1,2-nonadiol, 1,9-nonadiol, 1,10-decanediol, 1,2-decanediol, 1,2-undecanediol, 1,11-undecanediol, 1,12-dodecanediol, 1,2-dodecanediol, diethylene glycol, dipropylene glycol, triethylene glycol, tripropylene glycol, tetraethylene glycol, tetrapropylene glycol, pentaethylene glycol, pentapropylene glycol, hexaethylene glycol, hexapropylene glycol, heptaethylene glycol, heptapropylene glycol, octaethylene glycol, octapropylene glycol, nonaethylene glycol, nonapropylene glycol, decaethylene glycol, decapropylene glycol, cis- or trans-1,2-cylopentanediol, cis- or trans-1,3-cylopentanediol, cis- or trans-1,2-cylohexanediol, cis- or trans-1,3-cylohexanediol, cis- or trans-1,4-cylohexanediol, cis- or trans-1,2-cyloheptanediol, cis- or trans-1,3-cyloheptanediol, cis- or trans-1,4-cyloheptanediol, 1,2,3-cyclopentanetriol, 1,2,4-cyclopentanetriol, 1,2,3-cyclohexanetriol, 1,2,4-cyclohexanetriol, 1,2,3-cyloheptanetriol, 1,2,4-cyloheptanetriol, 1,2,3-propanetriol, 3-ethyl-2-hydroxymethyl-1,3-propanediol, 2-hydroxymethyl-2-methyl-1,3-propanediol, 2-hydroxymethyl-2-methyl-1,3-propanediol, 1,2,3-butanetriol, 1,2,4-butanetriol, 2-methyl-1,2,3-butanetriol, 2-methyl-1,2,4-butanetriol, 1,2,3-pentanetriol, 1,2,4-pentanetriol, 1,2,5-pentanetriol, 2,3,4-pentanetriol, 1,3,5-pentanetriol, 3-methyl-1,3,5-pentanetriol, 1,2,3-hexanetriol, 1,2,4-hexanetriol, 1,2,5-hexanetriol, 1,2,6-hexanetriol, 2,3,4-hexanetriol, 2,3,5-hexanetriol, 1,2,3-heptanetriol, 1,2,7-heptanetriol, 1,2,3-octanetriol, 1,2,8-octanetriol, 1,2,3-nonatriol, 1,2,9-nonatriol, 1,2,3-decanetriol, 1,2,10-decanetriol, 1,2,3-undecanetriol, 1,2,11-undecanetriol, 1,2,3-dodecanetriol, 1,1,12-dodecanetriol, 2,2,-bis(hydroxymethyl)-1,3-propanediol, 1,2,3,4-butanetetraol, 1,2,3,4-pentanetetraol, 1,2,3,5-pentanetetraol, 1,2,3,4-hexanetetraol, 1,2,3,6-hexanetetraol, 1,2,3,4-heptanetetraol, 1,2,3,7-heptanetetraol, 1,2,3,4-octanetetraol, 1,2,3,8-octanetetraol, 1,2,3,4-nonanetetraol, 1,2,3,9-nonanetetraol, 1,2,3,4-decanetetraol, 1,2,3,10-decanetetraol, trimethylolpropanol, pentaerythritol, sugar alcohols such as mannitol, sorbitol or arabitol, hexanehexol, 1,2,3,4,5-pentanepentol and 1,2,3,4,5,6-hexanehexaol. In some embodiments, the polyol is 1,2,3-propanetriol (also referred to as glycerol).

In some embodiments, any of the compositions or reaction mixtures of the present disclosure include dimethyl sulfoxide.

In some embodiments, any of the compositions or reaction mixtures of the present disclosure include one or more salts or surfactants.

### Methods of Tagmentation in the Presence of a Histone-Like Protein

The present disclosure also provides methods of tagmenting DNA in the presence of one or more histone-like proteins. In general, the methods comprise forming a fragmentation reaction mixture, and heating the fragmentation reaction mixture at a predetermined temperature for a predetermined amount of time. Different embodiments of this general method are described herein.

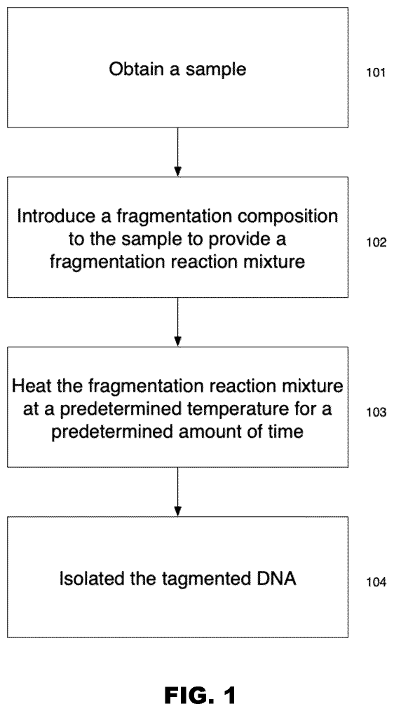

With reference to FIG. 1, a sample is first obtained (step 101). In some embodiments, the sample comprises double stranded DNA. In some embodiments, the double stranded DNA is genomic DNA. In some embodiments, the double stranded DNA is genomic DNA derived from a mammalian subject, e.g., a human patient. In some embodiments, the double stranded DNA is derived from a tumor sample. In some embodiments, the double stranded DNA is cDNA or ctDNA.

In some embodiments, a fragmentation composition is added (step 102) to the obtained sample to provide a fragmentation reaction mixture. As noted herein, the fragmentation composition may comprise one or more histone-like proteins, one or more transposition systems, and one or more optional additional components. In some embodiments, the obtained sample is allowed time to mix with the fragmentation composition before it is heated. In some embodiments, the obtained sample is allowed to mix with the fragmentation composition for about 30 seconds, about 1 minute, about 2 minutes, about 4 minutes, about 5 minutes, about 6 minutes, about 10 minutes, about 20 minutes, or about 30 minutes before the it is heated.

Subsequently, the fragmentation reaction mixture is heated at a predetermined temperature for a predetermined amount of time (step 103), i.e., a tagmentation reaction is carried out by heating the fragmentation reaction mixture. In some embodiments, the tagmentation reaction can be carried out at temperature ranging from about 25° C. to about 70° C., from about 37° C. to about 65° C., from about 50° C. to about 65° C., or from about 50° C. to about 60° C. In some embodiments, the tagmentation reaction can be carried out at a temperature of about 37° C., about 40° C., about 45° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., or about 65° C. In some embodiments, the tagmentation reaction can be carried out for a time period ranging from between about 30 seconds to about 10 minutes; from about 1 minute to about 8 minutes; from about 2 minutes to about 8 minutes; from about 3 minutes to about 7 minutes; or from about 4 minutes to about 6 minutes. In some embodiments, the tagmentation reaction may be carried out for about 2 minutes; for about 3 minutes; for about 4 minutes; for about 5 minutes; or for about 6 minutes. Methods of tagmenting DNA and additional reaction conditions using transposition systems are described in U.S. Pat. No. 9,080,211 and in United States Patent Publication No. US2010/0120098, the disclosures of which are hereby incorporated by reference herein in their entireties.

Next, the tagmented DNA is isolated from the fragmentation reaction mixture (step 104). In some embodiments, the isolation of the tagmented DNA comprises: (a) capturing generated DNA fragments onto beads using capture probes; (b) flushing impurities and non-targeted fragments from the reaction mixture; and (c) eluting or releasing the captured DNA fragments from the beads or capture probes.

In some embodiments, the tagmented DNA has a mean fragment size ranging from between about 200 bp to about 400 bp, from about 220 bp to about 360 bp, from about 240 bp to about 330 bp, or from about 250 bp to about 300 bp.

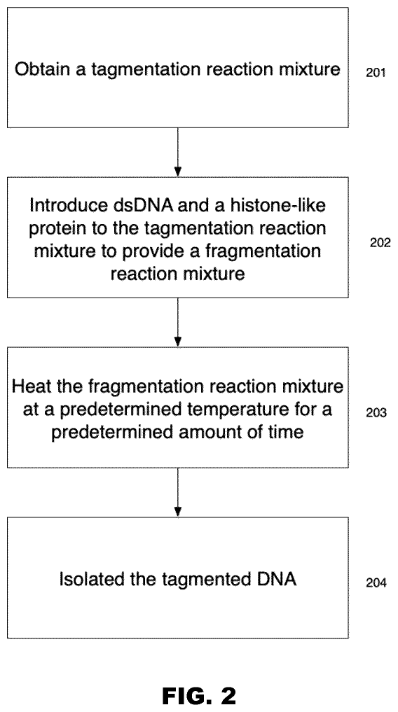

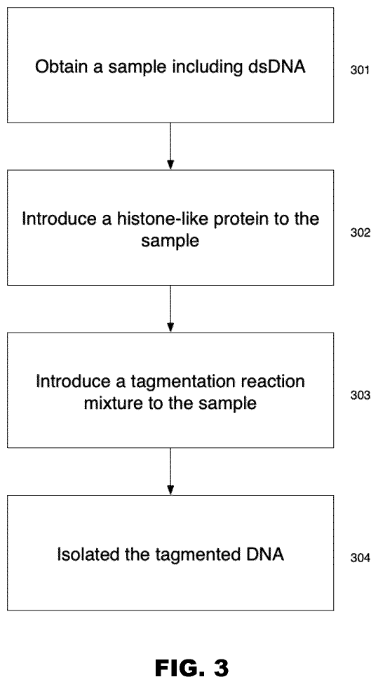

An alternative method of tagmenting DNA is illustrated in FIG. 2. In this embodiment, a tagmentation reaction mixture is first obtained (step 201). Subsequently, double stranded DNA and a histone-like protein are introduced to the tagmentation reaction mixture to form a fragmentation reaction mixture (step 202). In some embodiments, the double stranded DNA and the histone-like protein are added sequentially (see FIG. 3, namely steps 302 and 303, which may be carried out in any order). In other embodiments, the double stranded DNA and the histone-like protein are added simultaneously. In yet other embodiments, the double stranded DNA and the histone-like protein are first combined to form a DNA-histone-like protein solution, which is subsequently added to the tagmentation reaction mixture to form the fragmentation reaction mixture. Next, the fragmentation reaction mixture is heated at a predetermined temperature for a predetermined amount of time (step 103). Following tagmentation, the tagmented DNA is isolated (step 104). In some embodiments, the tagmented DNA is “cleaned-up” prior to amplification to remove impurities. In some embodiments, the “clean-up” utilized functionalized beads, such as KAPA PureBeads (KAPA Biosystems, Inc., Wilmington, Mass).

Following tagmentation and isolated of the tagmented DNA, the tagmented DNA may be optionally amplified (step 401) and/or sequenced (step 402). In some embodiments, sequencing is carried out using next generation sequencing.

### Kit Components

The present disclosure also provides for kits including any of the compositions or reaction mixtures described herein. In some embodiments, the kits disclosed herein may optionally contain other components including, but not limited to, reagents for conducting a polymerase chain reaction (e.g., PCR primers, a polymerase, buffer, nucleotides etc.). The various components of the kits of the present disclosure may be present in separate containers or certain compatible components may be pre-combined into a single container, as desired.

In some embodiments, any of the kits of the present disclosure may further include one or reagents for conducting a polymerase chain reaction (PCR). In some embodiments, the PCR reagents include deoxynucleoside triphosphates (dNTPs), in particular all of the four naturally-occurring deoxynucleoside triphosphates (dNTPs). In some embodiments, the PCR reagents include deoxyribonucleoside triphosphate molecules, including all of dATP, dCTP, dGTP, dTTP. In some embodiments, the PCR reagents also include compounds useful in assisting the activity of the nucleic acid polymerase. For example, in some embodiments, the PCR reagent include a divalent cation, e.g., magnesium ions. In some embodiments, the magnesium ions are provided in the form of magnesium chloride, magnesium acetate, or magnesium sulfate. In some embodiments, the PCR reagents further include a buffer or buffer solution, including any of the buffers recited herein.

In some embodiments, any of the kits of the present disclosure may further include at least one polymerase, modified polymerase, or thermostable polymerase. As used herein, the term “polymerase” refers to an enzyme that performs template-directed synthesis of polynucleotides. A DNA polymerase can add free nucleotides only to the 3′ end of the newly forming strand. This results in elongation of the newly forming strand in a 5′-3′ direction. No known DNA polymerase is able to begin a new chain (de novo). DNA polymerase can add a nucleotide only on to a pre-existing 3′—OH group, and, therefore, needs a primer at which it can add the first nucleotide. Non-limiting examples of polymerases include prokaryotic DNA polymerases (e.g. Pol I, Pol II, Pol III, Pol IV and Pol V), eukaryotic DNA polymerase, archaeal DNA polymerase, telomerase, reverse transcriptase and RNA polymerase. Reverse transcriptase is an RNA-dependent DNA polymerase which synthesizes DNA from an RNA template. The reverse transcriptase family contain both DNA polymerase functionality and RNase H functionality, which degrades RNA base-paired to DNA. RNA polymerase, is an enzyme that synthesizes RNA using DNA as a template during the process of gene transcription. RNA polymerase polymerizes ribonucleotides at the 3′ end of an RNA transcript.

In some embodiments, suitable polymerases may be derived from: archaea (e.g., Thermococcus litoralis (Vent, GenBank: AAA72101), Pyrococcus furiosus (Pfu, GenBank: D12983, BAA02362), Pyrococcus woesii, Pyrococcus GB-D (Deep Vent, GenBank: AAA67131), Thermococcus kodakaraensis KODI (KOD, GenBank: BD175553, BAA06142; Thermococcus sp. strain KOD (Pfx, GenBank: AAE68738)), Thermococcus gorgonarius (Tgo, Pdb: 4699806), Sulfolobus solataricus (GenBank: NC002754, P26811), Aeropyrum pernix (GenBank: BAA81109), Archaeglobus fulgidus (GenBank: 029753), Pyrobaculum aerophilum (GenBank: AAL63952), Pyrodictium occultum (GenBank: BAA07579, BAA07580), Thermococcus 9 degree Nm (GenBank: AAA88769, Q56366), Thermococcus fumicolans (GenBank: CAA93738, P74918), Thermococcus hydrothermalis (GenBank: CAC18555), Thermococcus sp. GE8 (GenBank: CAC12850), Thermococcus sp. JDF-3 (GenBank: AX135456; WO0132887), Thermococcus sp. TY (GenBank: CAA73475), Pyrococcus abyssi (GenBank: P77916), Pyrococcus glycovorans (GenBank: CAC12849), Pyrococcus horikoshii (GenBank: NP 143776), Pyrococcus sp. GE23 (GenBank: CAA90887), Pyrococcus sp. ST700 (GenBank: CAC 12847), Thermococcus pacificus (GenBank: AX411312.1), Thermococcus zilligii (GenBank: DQ3366890), Thermococcus aggregans, Thermococcus barossii, Thermococcus celer (GenBank: DD259850.1), Thermococcus profundus (GenBank: E14137), Thermococcus siculi (GenBank: DD259857.1), Thermococcus thioreducens, Thermococcus onnurineus NA1, Sulfolobus acidocaldarium, Sulfolobus tokodaii, Pyrobaculum calidifontis, Pyrobaculum islandicum (GenBank: AAF27815), Methanococcus jannaschii (GenBank: Q58295), Desulforococcus species TOK, Desulforococcus, Pyrolobus, Pyrodictium, Staphylothermus, Vulcanisaetta, Methanococcus (GenBank: P52025) and other archaeal B polymerases, such as GenBank AAC62712, P956901, BAAA07579)), thermophilic bacteria Thermus species (e.g., flavus, ruber, thermophilus, lacteus, rubens, aquaticus), Bacillus stearothermophilus, Thermotoga maritima, Methanothermus fervidus, KOD polymerase, TNA1 polymerase, Thermococcus sp. 9 degrees N-7, T4, T7, phi29, Pyrococcus furiosus, P. abyssi, T. gorgonarius, T. litoralis, T. zilligii, T. sp. GT, P. sp. GB-D, KOD, Pfu, T. gorgonarius, T. zilligii, T. litoralis and Thermococcus sp. 9N-7 polymerases.

As used herein, the term “modified DNA polymerase” refers to a DNA polymerase originated from another (i.e., parental) DNA polymerase and contains one or more amino acid alterations (e.g., amino acid substitution, deletion, or insertion) compared to the parental DNA polymerase. In some embodiments, a modified DNA polymerases of the disclosure is originated or modified from a naturally-occurring or wild-type DNA polymerase. In some embodiments, a modified DNA polymerase of the disclosure is originated or modified from a recombinant or engineered DNA polymerase including, but not limited to, chimeric DNA polymerase, fusion DNA polymerase or another modified DNA polymerase. Typically, a modified DNA polymerase has at least one changed phenotype compared to the parental polymerase. Examples of modified polymerases are described in United States Patent Application Publication No. 2016/0222363, the disclosure of which is incorporated by reference herein in its entirety.

As used herein, the term “thermostable polymerase” refers to an enzyme that is stable to heat, is heat resistant, and retains sufficient activity to effect subsequent polynucleotide extension reactions and does not become irreversibly denatured (inactivated) when subjected to the elevated temperatures for the time necessary to effect denaturation of double-stranded nucleic acids. The heating conditions necessary for nucleic acid denaturation are well known in the art and are exemplified in, e.g., U.S. Pat. Nos. 4,683,202, 4,683,195, and 4,965,188, which are incorporated herein by reference. As used herein, a thermostable polymerase is suitable for use in a temperature cycling reaction such as the polymerase chain reaction (“PCR”), a primer extension reaction, or an end-modification (e.g., terminal transferase, degradation, or polishing) reaction. Irreversible denaturation for purposes herein refers to permanent and complete loss of enzymatic activity. For a thermostable polymerase, enzymatic activity refers to the catalysis of the combination of the nucleotides in the proper manner to form polynucleotide extension products that are complementary to a template nucleic acid strand. Thermostable DNA polymerases from thermophilic bacteria include, e.g., DNA polymerases from Thermotoga maritima, Thermus aquaticus, Thermus thermophilus, Thermus flavus, Thermus filiformis, Thermus species sps17, Thermus species Z05, Thermus caldophilus, Bacillus caldotenax, Thermotoga neopolitana, Thermosipho africanus, and other thermostable DNA polymerases disclosed above.

In some embodiments, a polymerase may be a modified naturally occurring Type A polymerase. A further embodiment of the invention generally relates to a method wherein a modified Type A polymerase, e.g., in a primer extension, end-modification (e.g., terminal transferase, degradation, or polishing), or amplification reaction, may be selected from any species of the genus Meiothermus, Thermotoga, or Thermomicrobium. Another embodiment of the invention generally pertains to a method wherein the polymerase, e.g., in a primer extension, end-modification (e.g., terminal transferase, degradation or polishing), or amplification reaction, may be isolated from any of Thermus aquaticus (Taq), Thermus thermophilus, Thermus caldophilus, or Thermus filiformis. A further embodiment of the invention generally encompasses a method wherein the modified Type A polymerase, e.g., in a primer extension, end-modification (e.g., terminal transferase, degradation, or polishing), or amplification reaction, may be isolated from Bacillus stearothermophilus, Sphaerobacter thermophilus, Dictoglomus thermophilum, or Escherichia coli. In another embodiment, the invention generally relates to a method wherein the modified Type A polymerase, e.g., in a primer extension, end-modification (e.g., terminal transferase, degradation, or polishing), or amplification reaction, may be a mutant Taq-E507K polymerase. Another embodiment of the invention generally pertains to a method wherein a thermostable polymerase may be used to effect amplification of the target nucleic acid.

In some embodiments, any of the kits of the present disclosure may further include a ligase. In some embodiments, the ligase is a DNA ligase. In some embodiments, the ligase is a thermostable single stranded RNA or DNA ligase such as the Thermophage Ligase or its derivatives such as Circligase™ and Circligase™ II (Epicentre Tech., Madison, Wise.). In other embodiments, the ligase is a T4 ligase.

In addition to above-mentioned components, the subject kits may further include instructions for using the components of the kit to practice the subject methods, i.e., to instructions for library preparation. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging). In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded.

In some embodiments, any one of the kits of the present disclosure may include a sequencing device, such as a sequencing device for “next generation sequencing.” The term “next generation sequencing” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Some examples of next generation sequencing techniques include, but are not limited to, sequencing by synthesis, sequencing by ligation, and sequencing by hybridization. These technologies produce shorter reads (anywhere from about 25-about 500 bp) but many hundreds of thousands or millions of reads in a relatively short time.

Examples of such sequencing devices available from Illumina (San Diego, CA) include, but are not limited to iSEQ, MiniSEQ, MiSEQ, NextSEQ, NoveSEQ. It is believed that the Illumina next-generation sequencing technology uses clonal amplification and sequencing by synthesis (SBS) chemistry to enable rapid sequencing. The process simultaneously identifies DNA bases while incorporating them into a nucleic acid chain. Each base emits a unique fluorescent signal as it is added to the growing strand, which is used to determine the order of the DNA sequence.

A non-limiting example of a sequencing device available from ThermoFisher Scientific (Waltham, MA) includes the Ion Personal Genome Machine™ (PGM™) System. It is believed that Ion Torrent sequencing measures the direct release of H+ (protons) from the incorporation of individual bases by DNA polymerase. A non-limiting example of a sequencing device available from Pacific Biosciences (Menlo Park, CA) includes the PacBio Sequel Systems. A non-limiting example of a sequencing device available from Roche (Pleasanton, CA) is the Roche 454.

Next-generation sequencing methods may also include nanopore sequencing methods. In general, three nanopore sequencing approaches have been pursued: strand sequencing in which the bases of DNA are identified as they pass sequentially through a nanopore, exonuclease-based nanopore sequencing in which nucleotides are enzymatically cleaved one-by-one from a DNA molecule and monitored as they are captured by and pass through the nanopore, and a nanopore sequencing by synthesis (SBS) approach in which identifiable polymer tags are attached to nucleotides and registered in nanopores during enzyme-catalyzed DNA synthesis. Common to all these methods is the need for precise control of the reaction rates so that each base is determined in order.

Strand sequencing requires a method for slowing down the passage of the DNA through the nanopore and decoding a plurality of bases within the channel; ratcheting approaches, taking advantage of molecular motors, have been developed for this purpose. Exonuclease-based sequencing requires the release of each nucleotide close enough to the pore to guarantee its capture and its transit through the pore at a rate slow enough to obtain a valid ionic current signal. In addition, both of these methods rely on distinctions among the four natural bases, two relatively similar purines and two similar pyrimidines. The nanopore SBS approach utilizes synthetic polymer tags attached to the nucleotides that are designed specifically to produce unique and readily distinguishable ionic current blockade signatures for sequence determination. In some embodiments, sequencing of nucleic acids comprises via nanopore sequencing comprises: preparing nanopore sequencing complexes and determining polynucleotide sequences. Methods of preparing nanopores and nanopore sequencing are described in U.S. Patent Application Publication No. 2017/0268052, and PCT Publication Nos. WO2014/074727, WO2006/028508, WO2012/083249, and WO/2014/074727, the disclosures of which are hereby incorporated by reference herein in their entireties. In some embodiments, tagged nucleotides may be used in the determination of the polynucleotide sequences (see, e.g., PCT Publication No. WO/2020/131759, WO/2013/191793, and WO/2015/148402, the disclosures of which are hereby incorporated by reference herein in their entireties).

In some embodiments, any one of the kits of the present disclosure may include software for analyzing obtained sequencing data. Analysis of the data generated by sequencing is generally performed using software and/or statistical algorithms that perform various data conversions, e.g., conversion of signal emissions into base calls, conversion of base calls into consensus sequences for a nucleic acid template, etc. Such software, statistical algorithms, and the use of such are described in detail, in U.S. Patent Application Publication Nos. 2009/0024331 2017/0044606 and in PCT Publication No. WO/2018/034745, the disclosures of which are hereby incorporated by reference herein in their entireties.

### Example

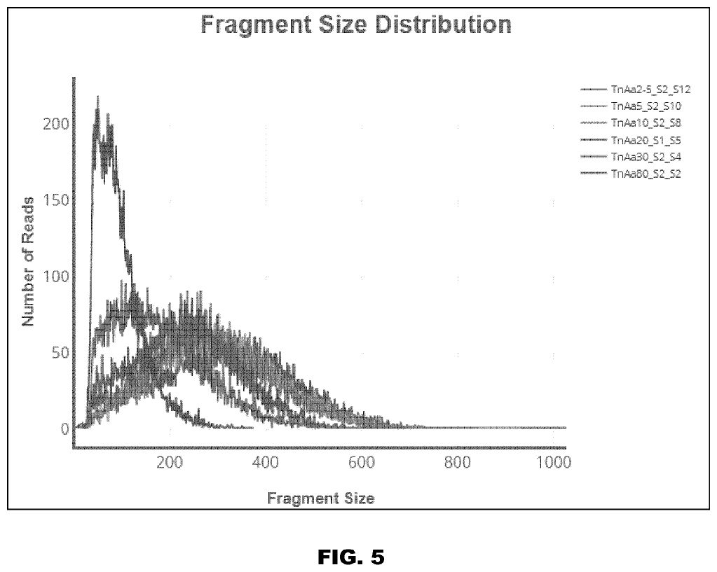

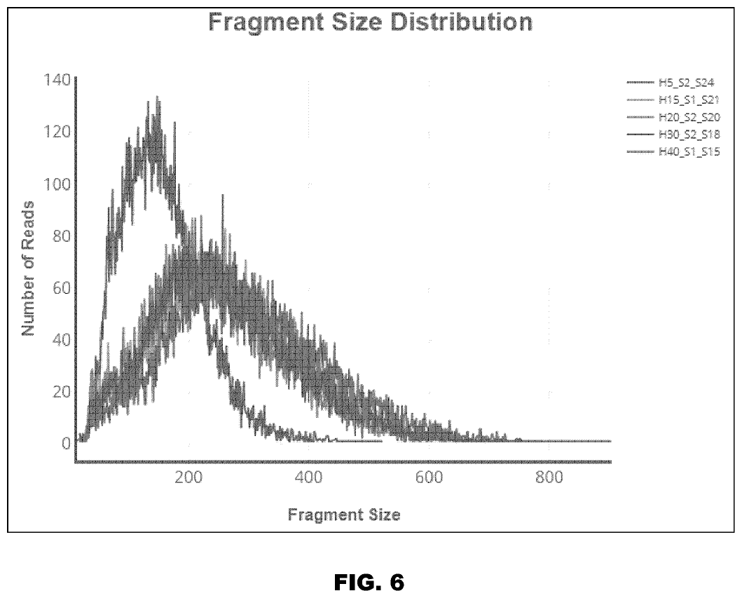

The tagmentation of DNA in the presence of histones-like proteins was demonstrated using E. coli DNA 10 ng (mg1655) and the transposase TnAa (Tn5L1) at a stock concentration of 180 ng/μL. The experiment was designed to demonstrate that larger fragments could be obtained by the addition of a histone-like protein without using dilution of the transposition system concentration to control for fragment size. Tagmentation reaction setups including between 2.5 ng and 80 ng total input of transposition system (TnAa) yielded a similar range of library sizes compared to tagmentation in the presence of a histone-like protein (compare FIGS. 5 and 6).

The components of the reaction setups are provided below.

E. coli DNA 10 ng (mg1655) was tagmented and sequencing libraries were prepared with the following TnAa (Tn5L1) enzyme concentrations: 2.5 ng (0.625 ng/μL), 5 ng (1.25 ng/μL), 10 ng (2.5 ng/μL), 20 ng (5 ng/μL), 30 ng (7.5 ng/μL), and 80 ng (20 ng/μL). The TnAa transposase included within the transposition system has SEQ ID NO: 5.

### Tagmentation Reaction Mixture

| Component | Cons. | Volume | |
|---|---|---|---|
| LMW Buffer | 2x | 10 | μL |
| MnCl | 10 mM | 2 | μL |
| DNA | | 4 | μL |
| Enzyme | | 4 | μL |
| (transposition | | | |
| system) and Dilution | | | |
| Buffer | | | |
| Total | | 20 | μL |

### LMW Buffer (2X):

| Component | Cons. |
|---|---|
| Glycerol | 40% |
| Tris-Acetate (pH | 50 mM |
| 7.5) | |
| DMSO | 30 mM |
| (dimethylsulfoxide) | |

### Dilution Buffer

| Component | Cons. |
|---|---|
| Formulation | 50% |
| Buffer | |
| PIPES, pH | 20 mM |
| 7.5 | |
| Glycerol | 13.13% |

### Storage Buffer

| Component | Cons. |
|---|---|
| Formulation | 50% |
| Buffer | |
| PIPES, pH | 20 |
| 7.5 | |
| Glycerol | 26.25 |

E. coli DNA 10 ng (mg1655) was tagmented in the presence of a histone-like protein (HphA). The fragmentation reaction mixture included a transposition system at a concentration of 180 ng/μL; and histone-like protein in amounts of 40 ng (20 ng/μL), 30 ng (15 ng/μL), 20 ng (10 ng/μL), 15 ng (7.5 ng/μL), 5 ng (2.5 ng/μL), an Ong. The DNA concentration was held constant at 5 ng/μL in each experiment.

### Fragmentation Reaction Mixture

| Component | Cons. | Volume | |
|---|---|---|---|
| LMW Buffer | 2x | 10 | μL |
| MnCl | 10 mM | 2 | μL |
| DNA + HphA protein | | 2 μL + 2 μL | |
| Enzyme (transposition system) | | 4 | μL |
| Total | | 20 | μL |

The tagmentation reaction conditions were as follows:
52° C. for 5 min
5 μL Stop solution added (0.25% SDS) [What else is included within the stop solution?]
0.8× Kapa pure bead clean-up
Elute in 22 μL 10mMTris

Amplification was carried around following the preparation of the tagmented DNA using the following parameters:

### Tagmentation Amplification

| | Cons. | Volume | |
|---|---|---|---|
| Tagmented DNA | | 20 | ul |
| N5XX | 2.5 uM | 5 | ul |
| N7XX | 2.5 uM | 5 | ul |
| Hifi Buffer | 5x | 10 | ul |
| Hifi Enzyme | | 1 | ul |
| H2O | | 7.5 | ul |
| dNTP | | 1.5 | ul |
| total | | 50 | ul |

| | | |
|---|---|---|
| Temperature | Time | Cycle |

| | | | |
|---|---|---|---|
| 72° C. | 3 | min | 1 |
| 98° C. | 30 | sec | |
| 98° C. | 10 | sec | 12 |
| 63° C. | 30 | sec | |
| 72° C. | 3 | min | 1 |

The samples were then sequenced on an Ilumina MiniSeq using the MiniSeq System Mid-Output Kit (2×150 bp). Sequencing reads were quality filtered, sampled and evaluated based on insert size and start site sequence bias of the insert.

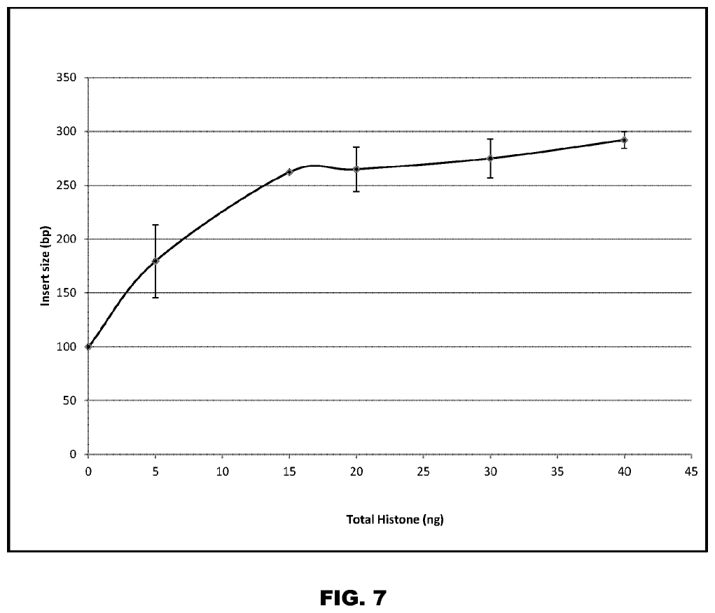

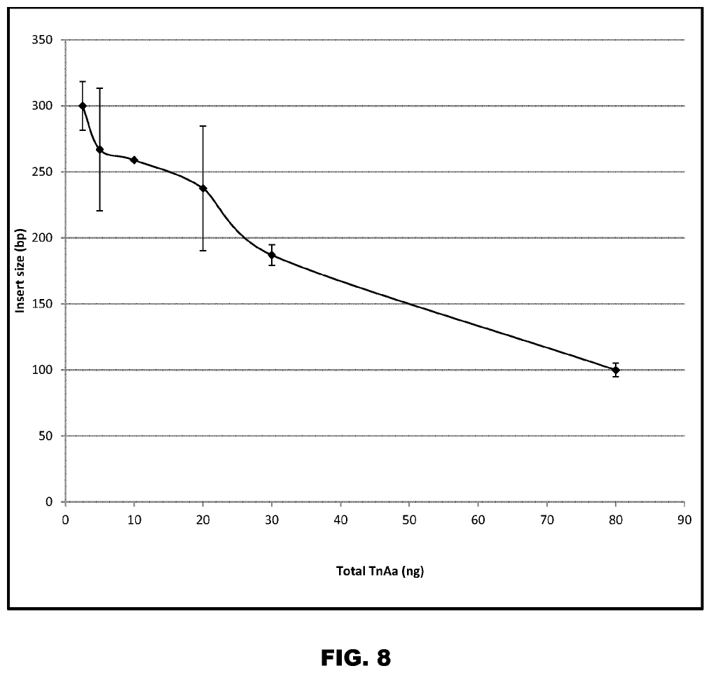

The mean insert sizes of the tagmentation libraries increased with an increase in the amount of histone-like protein added (see FIG. 7). The insert size plateaued at an insert size of 300 bp with 20 ng to 40 ng histone-like protein added (see FIG. 7). The start site bias was not influenced by the amount of histone added to the tagmentation reaction (data not shown). The mean insert sizes of the libraries decreased with an increase in the TnAa enzyme (see FIG. 8). The results indicated that it was possible to obtain larger insert sizes with the use of stock concentration (180 ng/μL) of the transposase enzyme with a mean insert size of up to 300 bp.

Applicants concluded that the use of histone-like proteins in the tag mention reaction has the following undermentioned advantages: (i) eliminate the need for precise transposase enzyme dilutions; (ii) potentially reduce the observed stability issues of diluted TnAa enzyme; and (iii) potentially add some flexibility in DNA concentration.

1. A composition comprising a histone-like protein and a transposition system, wherein the histone-like protein is an archaeal histone-like protein derived from one of a Thermococcus or a Pyrococcus.

2. The composition of claim 1, wherein the histone-like protein comprises an amino acid sequence having at least 85% identity to SEQ ID NOS: 1 or 2.

3. The composition of claim 1-2, wherein a concentration of the histone-like protein in the composition ranges from between about 2.5 ng/μL to about 25 ng/μL.

4. The composition of claim 1-3, wherein the transposition system comprises a transposase, and one or more adapters.

5. The composition of claim 1,-4 wherein the transposition system comprises a hyperactive Tn5 transposase and a Tn5-type transposase recognition site.

6. The composition of claim 5, wherein the transposition system further comprises one or more oligonucleotides.

7. The composition of claim 1-6, wherein a concentration of the transposition system in the composition ranges from between about 150 ng/μL to about 200 ng/μL.

8. The composition of claim 1-7, further comprising double-stranded DNA, wherein a concentration of the double-stranded DNA in the composition ranges from between about 2 ng/μL to about 8 ng/μL.

9. The composition of claim 1-7, further comprising double-stranded DNA.wherein a ratio of a concentration of the histone-like protein to a concentration of DNA in the composition ranges from between about 0.5:1 to about 5:1.

10. The composition of claim 1-9, further comprising a divalent cation selected from the group consisting of Co²⁺, Mn²⁺, Mg²⁺, Cd²⁺, and Ca²⁺.

11. The composition of claim 1-10, further comprising a low molecular weight (LMW) buffer, which comprises tris-acetate, glycerol, and DMSO.

12. A kit comprising:
(a) a first container comprising a histone-like protein; and
(b) a second container comprising a transposition system;
wherein the histone-like protein is an archaeal histone-like protein derived from one of a Thermococcus or a Pyrococcus; and
wherein the transposition system comprises a transposase and adapters;
wherein the transposition system comprises: (i) TnAa; or (ii) a hyperactive Tn5 transposase and a Tn5-type transposase recognition site; and
wherein the transposition system further comprises one or more oligonucleotides.

13. A method of tagmenting double-strand DNA comprising:
(a) obtaining a sample comprising double-stranded DNA;
(b) introducing a fragmentation composition comprising a histone-like protein and a transposition system to the obtained sample to provide a fragmentation reaction mixture;
(c) heating the fragmentation reaction mixture for a predetermined amount of time at a predetermined temperature; and
(d) isolating the tagmented DNA from the fragmentation reaction mixture.

14. A method for processing a sample including genomic material comprising:
(a) obtaining a tagmentation reaction mixture including comprising a transposition system;
(b) introducing double-stranded DNA and a histone-like protein to the tagmentation reaction mixture to provide a fragmentation reaction mixture; and
(c) heating the fragmentation reaction mixture to a predetermined temperature for a predetermined amount of time.

15. A method for processing a sample including genomic material comprising:
(a) obtaining a tagmentation reaction mixture including a buffer and a transposition system;
(b) introducing a solution comprising one or more nucleosome-like structures to the tagmentation reaction mixture to provide a fragmentation reaction mixture, wherein the one or more nucleosome-like structures include double-stranded DNA wound or wrapped around one or more histone-like proteins; and
(c) heating the fragmentation reaction mixture to a predetermined temperature for a predetermined amount of time.

16. A method for processing a sample including genomic material, comprising:
(a) obtaining a sample in a reaction vessel, the sample including double stranded DNA material;
(b) introducing a histone-like protein to the reaction vessel to provide a DNA-histone-like protein solution;
(c) introducing a transposition system to the DNA-histone-like protein solution to provide a fragmentation reaction mixture; and
(d) heating the fragmentation reaction mixture to a predetermined temperature for a predetermined amount of time.

17. Double stranded DNA fragments having a size ranging from between about 250 to about 300 bp, wherein the double stranded DNA fragments are prepared by:
(a) obtaining a sample comprising double-stranded DNA;
(b) introducing a fragmentation composition comprising a histone-like protein, and a transposition system to the sample, to provide a fragmentation reaction mixture; and
(c) heating the fragmentation reaction mixture for a predetermined amount of time at a predetermined temperature.
